SEQUENCE LISTING (13 sequences)

SEQ ID NO: 1 - DNA, 1491 bp, Clostridium hiranonis
  source 1..1491: mol_type = genomic DNA; note = Clostridium hiranonis DSM 13275 >CP036523 (of whole genome)
agagtttgatcctggctcaggatgaacgctggcggcgtgcctaacacatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactaggtgtcggaggttacccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaagccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttggagacacccgaagccgactatctaaccttttgggagaagtcgtcgaaggtggaatcaataactggggtgaagtcgtaacaaggtagccgtatcggaaggtgcggctggatcacct

SEQ ID NO: 2 - DNA, 1521 bp, Anaerostipes hadrus
  source 1..1521: mol_type = genomic DNA; note = Anaerostipes hadrus DSM 3319 >NR_117139.2
tttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctaccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccttatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggccttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacctcctt

SEQ ID NO: 3 - DNA, 1539 bp, Bacteroides stercoris
  source 1..1539: mol_type = genomic DNA; note = Bacteroides stercoris CC31F >NZ_ATFP00000000.1 (of whole genome)
gctccagaaaggaggtgttccagccgcaccttccggtacggctaccttgttacgacttagccccaatcaccagttttaccctaggacgctcctcgcggttacgtacttcaggtacccccggctttcatggcttgacgggcggtgtgtacaaggcccgggaacgtattcaccgcgccgtggctgatgcgcgattactagcgaatccagcttcatggagtcgggttgcagactccaatccgaactgagagaggctttcgggattagcaccacgtcgccgtgtagctgccttctgtaccccccattgtaacacgtgtgtagccccggacgtaagggccgtgctgatttgacgtcatccccaccttcctcacatcttacgacggcagtccgggtagagtccccagcataacctgatggtaactacccgtaagggttgcgctcgttatggcacttaagccgacacctcacggcacgagctgacgacaaccatgcagcaccttcacaactgtccgaagaaagaaccgtttccgattcagtcagttgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgctgaccgtatatcgccaacagcgagtattcatcgtttactgtgtggactaccagggtatctaatcctgtttgatacccacactttcgagcatcagcgtcagttacaatccagtaagctgccttcgcaatcggagttcttcgtgatatctaagcatttcaccgctacaccacgaattccgcctacctctgttgcactcaaggtcgccagtatcaactgcaattttacggttgagccgcaaactttcacaactgacttaacaacccgcctacgctccctttaaacccaataaatccggataacgctcggatcctccgtattaccgcggctgctggcacggagttagccgatccttattcatacggtacatacaaaaagccacacgtggctcactttattcccgtataaaagaagtttacaacccatagggcagtcatccttcacgctacttggctggttcagactctcgtccattgaccaatattcctcactgctgcctcccgtaggagtttggaccgtgtctcagttccaatgtgggggaccttcctctcagaacccctatccatcgtaggtttggtgggccgttaccccgccaactgcctaatggaacgcatccccatcgacaaccgaaattctttaatagtcatcccatgcgggaaaactatgccatccggtattaatctttctttcgaaaggctatcccagtgttgtcggcaggttggatacgtgttactcacccgtgcgccggtcgccatccatcaaagcaagctttgatgatgctgcccctcgacttgcatgtgttaagcctgtagctagcgttcatcctgagccaggatcaaactcttcattgtaaaag

SEQ ID NO: 4 - DNA, 1476 bp, Bacteroides stercoris
  source 1..1476: mol_type = genomic DNA; note = Bacteroides stercoris ATCC 43183 > NR_112943
tggctcaggatgaacgctagctacaggcttaacacatgcaagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggatgattattaaagaatttcggttgtcgatggggatgcgttccattaggcagttggcggggtaacggcccaccaaacctacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgactgaatcggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcgaggagcgtcctagggtaaaactggtgattggggctaagtcgtaacaaggtaacc

SEQ ID NO: 5 - DNA, 1610 bp, Phascolarctobacterium succinatutens
  source 1..1610: mol_type = genomic DNA; note = Phascolarctobacterium succinatutens YIT 12067 > GenBank: AB490811.1
gacgaacgctggcggcatgcctaacacatgcaagtcgaacggagaaagttcaacaccaagtatttcatccgctgaagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtaaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaagg

SEQ ID NO: 6 - DNA, 1514 bp, Clostridium bolteae
  source 1..1514: mol_type = genomic DNA; note = Clostridium bolteae ATCC BAA-613 > CP022464.2 (of whole genome)
gagagtttgatcctggctcaggatgaacgctggcggcgtgcctaacacatgcaagtcgaacgaagcaattgaaggaagttttcggatggaattcgattgactgagtggcggacgggtgagtaacgcgtggataacctgcctcacactgggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctgg

SEQ ID NO: 7 - DNA, 1538 bp, Megamonas hypermegale
  source 1..1538: mol_type = genomic DNA; note = Megamonas hypermegale strain NCTC10570 > LT906446.1 (of whole genome)
agagtttgatcctggctcaggacgaacgctggcggcgtgcttaacacatgcaagtcgaacggggtgtttatttcggtaaacaccaagtggcgaacgggtgagtaacgcgtaagcaatctaccttcaagatggggacaacacttcgaaaggggtgctaataccgaatgaatgagagatgaccgcatggatatttctctaaaggaggcctctgaaaatgcttccgcttgaagatgagcttgcgtctgattagctagttggtgagggtaaaggcccaccaaggcgacgatcagtagccggtctgagaggatgaacggccacattgggactgagacacggcccagactcctacgggaggcagcagtggggaatcttccgcaatgggcgaaagcctgacggagcaacgccgcgtgaacgaagaaggtcttaggatcgtaaagttctgttgttaggggcgaagggcaacattttgaataagggtgttgtttgacggtacttaacgaggaagccacggctaactacgtgccagcagccgcggtaatacgtaggcggcaagcgttgtccggaattattgggcgtaaagggagcgcaggcgggaagttaagcggactttaaaagtgcggggctcaaccccgtgagggggtccgaactgactttcttgagtgcaggagagggaagcggaattcctagtgtagcggtgaaatgcgtagatattaggaagaacaccagtggcgaaggcggctttctggactgtaactgacgctgaggctcgaaagctagggtagcgaacgggattagataccccggtagtcctagccgtaaacgatggatactaggtgtgggaggtatcgaccccttccgtgccggagttaacgcaataagtatcccgcctggggagtacggccgcaaggttgaaactcaaaggaattgacgggggcccgcacaagcggtggagtatgtggtttaattcgacgcaacgcgaagaaccttaccaagacttgacatcgactgacgtacttagagataagtatttttacttcggtaaacaggaagacaggtggtgcatggctgtcgtcagctcgtgtcgtgagatgttgggttaagtcccgcaacgagcgcaacccctatactatgttgccagcacgtaaaggtgggaactcatagtagactgccgcggacaacgcggaggaaggcggggatgacgtcaagtcatcatgccccttacgtcttgggctacacacgtactacaatgggatgaacagagggaagcgaagtcgcgaggcagagcggaaccctaaaagcatctctcagttcggattgcaggctgaaactcgcctacatgaagtcggaatcgctagtaatcgcaggtcagcatactgcggtgaatacgttcccgggccttgtacacaccgcccgtcacaccacgaaagtcattcacacccgaagccggctaagggccgaaaggaaccgaccgtcgaaggtgggggcgatgattggggtgaagtcgtaacaaggtagccgtatcggaaggtgcggctgga

SEQ ID NO: 8 - DNA, 1548 bp, Lactococcus lactis
  source 1..1548: mol_type = genomic DNA; note = Lactococcus lactis subsp. cremoris MG1363 > AM406671.1 (of whole genome)
tttatttgagagtttgatcctggctcaggacgaacgctggcggcgtgcctaatacatgcaagttgagcgctgaaggttggtacttgtaccgactggatgagcagcgaacgggtgagtaacgcgtggggaatctgcctttgagcgggggacaacatttggaaacgaatgctaataccgcataaaaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaacaaggtagccgtatcggaaggtgcggctggatcacctccttt

SEQ ID NO: 9 - DNA, 1529 bp, Clostridium scindens
  source 1..1529: mol_type = genomic DNA; note = > Clostridium scindens ATCC 35704 NCBI Reference Sequence: NR_028785.1 ncbi reference sequence: nr_028785.1
gagagtttgatcctggctcaggatgaacgctggcggcgtgcctaacacatgcaagtcgaacgaagcgcctggccccgacttcttcggaacgaggagccttgcgactgagtggcggacgggtgagtaacgcgtgggcaacctgccttgcactgggggataacagccagaaatggctgctaataccgcataagaccgaagcgccgcatggcgcggcggccaaagccccggcggtgcaagatgggcccgcgtctgattaggtagttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcattttggatgggcactctggagagactgccagggagaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagttggaatcgctagtaatcgcgaatcagaatgtcgcggtgaatacgttcccgggtcttgtacacaccgcccgtcacaccatgggagtcagtaacgcccgaagccggtgacccaacccgtaagggagggagccgtcgaaggtgggaccgataactggggtgaagtcgtaacaaggtagccgtatcggaaggtgcggctggatcacctccttc

SEQ ID NO: 10 - DNA, 1537 bp, Parabacteroides distasonis
  source 1..1537: mol_type = genomic DNA; note = Parabacteroides distasonis CL09T03C24 NCBI Reference Sequence: NR_074376.1
caatttaaacaacgaagagtttgatcctggctcaggatgaacgctagcgacaggcttaacacatgcaagtcgaggggcagcggggtgtagcaatacaccgccggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggttaggctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtagccgtaccggaaggtgcggctggaacacctcctttct

SEQ ID NO: 11 - DNA, 1526 bp, Eubacterium limosum
  source 1..1526: mol_type = genomic DNA; note = NZ_CP011914.1 Eubacterium limosum strain SA11 chromosome, complete genome
tttattgagagtttgatcctggctcaggacgaacgctggcggtatgcttaacacatgcaagtcgaacgagaagatcagttaagaaccttcgggggaataagtgattggaagtggcgaacgggtgagtaacgcgtgggtaacctgccctatggaaaggaatagcctcgggaaactgggagtaaagccttatattatggagagatcgcatggtcatttcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaaaacatgacggtacccaatgaggaagtcccggctaactacgtgccagcagccgcggtaatacgtaggggacaagcgttgtccggaatgactgggcgtaaagggcgcgtaggcggtctgataagtcagatgtgaaaggtaccggctcaaccggtgacgtgcatttgaaactgtcagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacaatcccagagatgggacgtttccttcgggaacagagagacaggtggtgcatggttgtcgtcagctcgtgtcgtgagatgttgggttaagtcccgcaacgagcgcaacccctgcctttagttgccagcattgagttgggcactctagagggactgccgtagacaatacggaggaaggtggggatgacgtcaaatcatcatgccccttatgacctgggctacacacgtgctacaatggtctgaacagagggccgcgaaaccgcgaggtgaagcaaatcccttaaaacagatcccagttcggattgcaggctgcaactcgcctgcataaagttggagttgctagtaatcgcggatcagaatgccgcggtgaatgcgttcccgggtcttgtacacaccgcccgtcacaccacgagagttggcaacacccgaagcccgtgagagaaccgtaaggactcagcggtcgaaggtggggctagtaattggggtgaagtcgtaacaaggtagccgtatcggaaggtgcggctggatcacctcctttcta

SEQ ID NO: 12 - DNA, 1455 bp, Clostridium sp.
  source 1..1455: mol_type = genomic DNA; note = Clostridium sp. SS2/1 16S sequence (identical to Anaerostipes hadrus DSM3319) Genebank accession AY305319.1
gatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtg

SEQ ID NO: 13 - DNA, 1521 bp, Lachnospira sp.
  source 1..1521: mol_type = genomic DNA; note = GL622435.1 Lachnospiraceae bacterium 5_1_63FAA (over 99% identical to Anaerostipes hadrus DSM3319)
agagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacct

(19) United States
(12) Patent Application Publication
(10) Pub. No.: US 2025/0387439 A1
(43) Pub. Date: Dec. 25, 2025

(54) MICROBIAL CONSORTIUM AND USES THEREOF

(71) Applicant: BIOMICA LTD, Rehovot (IL)

(72) Inventors: Shiri Meshner, Raanana (IL); Elran Haber, Kiryat Ono (IL); Shiri Eshar, Emek Sorek (IL); Osnat Tirosh, Ness-Ziona (IL); Sheerli Kruger Ben Shabat, Raanana (IL); Yehuda Ringel, Tel Aviv (IL); Omri Polonski, Kibbutz Givat Brenner (IL)

(73) Assignee: BIOMICA LTD, Rehovot (IL)

(21) Appl. No.: 19/304,673

(22) Filed: Aug. 20, 2025

Related U.S. Application Data
(62) Division of application No. 17/790,119, filed on Jun. 29, 2022, now abandoned.

(30) Foreign Application Priority Data
Dec. 31, 2019 (IL) 271775

(51) Int. Cl. A61K 35/742; A61K 35/00; A61K 35/741; A61K 35/744; A61P 1/00; A61P 37/06; C12N 1/20; C12R 1/01; C12R 1/145; C12R 1/46

(52) U.S. Cl. CPC A61K 35/742; A61K 35/741; A61K 35/744; A61P 1/00; A61P 37/06; C12N 1/205; A61K 2035/115; C12R 2001/01; C12R 2001/145; C12R 2001/46

(57) ABSTRACT

The present invention provides a microbial consortium comprising two or more microorganisms, compositions and kits comprising the same and uses thereof in methods of treating immune-related conditions.

### CROSS REFERENCE TO RELATED APPLICATIONS

This application is a divisional of U.S. application Ser. No. 17/790,119, filed Jun. 29, 2022, which is a U.S. National Phase of International Patent Application No. PCT/IL2020/051367, filed on Dec. 31, 2020, which claims priority to Israel Application No. 271775, filed Dec. 31, 2019. The entire contents of these applications are incorporated herein by reference in their entirety.

### SEQUENCE LISTING

The instant application contains a Sequence Listing which has been submitted electronically in XML format is hereby incorporated by reference in its entirety. Said XML copy, created on Aug. 19, 2025, is named 269002_000033_Seq_Listing.xml and is 35,195 bytes in size.

### TECHNOLOGICAL FIELD

The present discourse generally relates to consortium of microorganisms, compositions and kits comprising the same and uses thereof.

### BACKGROUND ART

References considered to be relevant as background to the presently disclosed subject matter are listed below:
[1] Lynch, Susan V., and Pedersen O., “The human intestinal microbiome in health and disease”, New England Journal of Medicine; (2016); 375; 2369-2379.
[2] International application publication No. WO2018187272
[3] U.S. Pat. No. 9,642,881
[4] U.S. Pat. No. 9,610,308
[5] US patent application No. 2019/0099455
[6] International application publication No. WO19/227085

Acknowledgement of the above references herein is not to be inferred as meaning that these are in any way relevant to the patentability of the presently disclosed subject matter.

### BACKGROUND

The human microbiome is a diverse multispecies population of more than trillion microorganisms including bacteria, fungi, archaea, and viruses that collectively play an important role in various physiological process within a host affecting human health and disease. For example, it was shown that the microbiome has the ability to increase energy extraction from food, to serve as a physical barrier to protect the host from external pathogens and to assist in the development of the host immune system [1].

Compositions of purified bacterial strains, and their use for treating disease were described [2-6].

### GENERAL DESCRIPTION

The present disclosure provides, in accordance with a first of its aspects a microbial consortium comprising two or more microorganisms, the two or more microorganisms are capable of modulating at least one phospholipid and/or at least one endocannabinoid and modulating at least one of (i) at least one short chain fatty acid (SCFA), (ii) lactate, (iii) secondary bile acid, (iv) a polysaccharide and (v) a glycosaminoglycan (GAG).

In accordance with some other aspects, the present disclosure provides a pharmaceutical composition comprising a microbial consortium comprising two or more microorganisms, the two or more microorganisms are capable of modulating at least one phospholipid and/or at least one endocannabinoid and modulating at least one of (i) at least one short chain fatty acid (SCFA), (ii) lactate, (iii) secondary bile acid, (iv) a polysaccharide and (v) a glycosaminoglycan (GAG).

Also provided by the present disclosure is a method of treating, preventing, ameliorating, reducing or delaying the onset of an inflammation condition in a human subject in need thereof comprising the step of administering to the subject an effective amount of a microbial consortium comprising two or more isolated microorganism or purified microorganism, the two or more microorganisms are capable of modulating at least one phospholipid and/or at least one endocannabinoid and modulating at least one of (i) at least one short chain fatty acid (SCFA), (ii) lactate, (iii) secondary bile acid, (iv) a polysaccharide and (v) a glycosaminoglycan (GAG).

Further provided herein is a kit comprising the microbial consortium comprising two or more isolated microorganism or purified microorganism, the two or more microorganisms are capable of modulating at least one phospholipid and/or at least one endocannabinoid and modulating at least one of (i) at least one short chain fatty acid (SCFA), (ii) lactate, (iii) secondary bile acid, (iv) a polysaccharide and (v) a glycosaminoglycan (GAG). In some embodiments, the kit comprising instructions for use in treating an inflammation condition.

### DESCRIPTION OF DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B are bar graph showing levels of arachidonic acid (AA) and anandamide (AEA), respectively, in RAW cells after treatment with A. hadrus, as detected in HPLC-MS/MS, results are represented as means±SEM, Y axis presents femtomoles of analyte per total protein in sample.

FIG. 2 is a graph showing lipocalin level (ng/g stool) in mice stool during the seven days of dextran sodium sulfate (DSS) administration, results are represented as means±SEM.

FIG. 3 is a bar graph showing disease severity in mice treated with bacterial consortia vs untreated mice following 7 days of DSS administration in mice.

FIG. 4 is a graph showing the effect of bacterial consortia treatment on Lipocalin-2 levels measured in mice stool throughout experiments days, data are presented as means±SEMs.

FIGS. 5A-5C are box plots showing the effect of bacterial consortia treatment on lipocalin-2 levels measured in mice stool in individual days: day 1, day 8 and day 14, the boxplots show the median with the interquartile range, and error bars are the 1.5 times interquartile range (whiskers).

FIG. 6 is a graph showing the effects of bacterial consortia treatment on clinical parameters of DSS-induced colitis as combined disease activity index (DAI score), data are presented as means±SEMs.

FIG. 7 is a graph showing mice spleen weights at study termination (day 20), boxplots show the median with the interquartile range, and error bars are the 1.5 times interquartile range (whiskers).

FIG. 8 is a graph showing mice colon lengths at study termination (day 20), boxplots show the median with the interquartile range, and error bars are the 1.5 times interquartile range (whiskers).

FIG. 9 is a graph showing survival probability calculated by Kaplen-Meier analysis and compared between the different groups.

### DETAILED DESCRIPTION

The gut microbiome includes a large number of diverse microorganisms that are capable of affecting a variety of physiological process within a host, including for example development and differentiation, activation or suppression of the host's immune system.

The present disclosure is based on a possible connection between the immune system and the gastrointestinal system and immune-related conditions such as inflammatory condition and is aimed at using specific microorganisms detected in, isolated from or purified from a microbiome, for example the gut microbiome, that contribute to modulation of immune system and hence may be used in preventing and treating immune-related conditions, specifically, inflammatory conditions.

To that end, the inventors have used an array of computational tools utilized to process high-throughput sequencing data and obtained high resolution detection and annotation of microbial genes and pathways as well as microbial taxa. This resulted in a mechanistic understanding of a relationship between an organisms' cellular processes (such as a microorganism) and various human cellular processes related to inflammatory conditions such as inflammatory conditions of the gut and immune function. During the analysis process several methods such as stringent statistical comparative analyses were applied to enable the identification and selection of specific microorganisms and microbial functions that differentiate between patients found to be diagnosed with an inflammatory disease in inflamed state versus non-inflamed state.

The attribution of a microorganism to a microbial consortium of the invention was done by considering the multiple functions of each one of the identified microorganisms and their combined functions. It was suggested that the selected microorganisms, are capable of reducing a pro-inflammatory effect and treating and/or preventing immune-related conditions such as inflammatory conditions, including, inter alia, inflammatory conditions of the gut, also by maintaining the integrity of the gut barrier.

It was also suggested that a unique and specific combinations of microorganisms identified computationally by the invention, may be administered, alone or preferably in specific combinations, to a subject in order to enrich the microbiome diversity and to treat or prevent an immune-related conditions such as inflammatory condition including, inter alia, an inflammatory condition of the gut. It was further suggested that the identified microorganisms may be used for diagnostic and prognostic purposes, for example for assessing responsiveness of a subject to treatment and for determining treatment protocols.

As such, the present disclosure provides a microbial consortium. The microbial consortium comprises two or more microorganisms that are collectively capable of modulating different physiological/biological processes (pathways) in the microorganisms including, inter alia, production of short chain fatty acids (SCFAs) and/or lactate. These products may be secreted from the microorganism to the host and in turn, modulate physiological/biological processes in a host. including, inter alia, activation or induction of Regulatory T cells (Tregs).

A physiological process as used herein encompasses physical and/or biological and/or chemical events in the microorganism and/or in the host that involve various functions and activities.

Surprisingly, it was found that microorganisms which were over-represented in patients diagnosed with a non-inflamed state of an inflammatory disease were capable of modulating host's endocannabinoid metabolism.

It was suggested that this modulation of host endocannabinoid metabolism is via a unique and novel mechanism which involves production of at least one phospholipid, such as phosphatidylcholine (PC), phosphatidylethanolamine (PE) or phosphatidylserine (PS), and a subsequent secretion from the microorganism to the host (possibly host gut). The at least one phospholipid may be used by a host as a precursor for endocannabinoid production.

Thus, the present disclosure provides in accordance with some aspects, a microbial consortium comprising two or more microorganisms, at least one of the two or more microorganisms is capable of modulating at least one phospholipid.

In the following text, when referring to the microbial consortium it is to be understood as also referring to the pharmaceutical compositions, kits and methods disclosed herein. Thus, whenever providing a feature with reference to the microbial consortium, it is to be understood as defining the same feature with respect to the pharmaceutical compositions, kits and methods, mutatis mutandis.

The microbial consortium as used herein refers to a mixture/cocktail of microorganisms, including at least one of a bacterium and/or an archaea. When referring to at least one microorganism it should be understood as referring to one microorganism species and/or strain as classified under common scientific classification. The microorganisms being the subject of the present disclosure are present in the human microbiome and thus can be isolated and/or purified from any microbiome, such as the human microbiome, by any known method in the art as also detailed below or purified from a biological material (e.g. fecal materials, such as feces or materials isolated from the various segments of the small and large intestines).

As such, the term microorganism used herein refers in accordance with some embodiments, an isolated microorganism, a purified microorganism, a recombinant microorganism or any combinations thereof.

In some embodiments, the microbial consortium comprises isolated microorganisms. In some embodiments, the microbial consortium comprises purified microorganisms. In some embodiments, the microbial consortium comprises recombinant microorganisms. In some embodiments, the microbial consortium comprises isolated microorganisms, purified microorganisms or any combination thereof.

It should be noted that the recombinant microorganisms of the present invention are microorganisms whose genetic makeup has been altered by deliberate introduction of new genetic elements. The recombinant microorganisms may maintain the functions (cellular processes) of the original microorganism.

In some embodiments, the microorganism is a live microorganism, provided as spores, heat-killed, non-living form of the microorganism, an extract of the organism, a component of the microorganism or any combination thereof.

In some embodiments, the microorganism is a live microorganism. In some other embodiments, the microorganism is provided as spores, heat-killed, non-living form of the microorganism. In some further embodiments, the microorganism is an extract of the organism. In yet some further embodiments, the microorganism is a component of the microorganism.

As described herein above, the computational analysis identified microorganisms which are over-represented (in higher abundance) in patients diagnosed with a non-inflamed state of an inflammatory disease such that at least one of these microorganisms was found to be capable of producing at least one phospholipid. In other words, the computational analysis described herein above have identified at least one microorganism that is capable of producing at least one phospholipid, specifically PE.

Phospholipids are a major component of all cell membranes and due to their amphiphilic nature, can form lipid bilayers. Phospholipids have a structure that generally consists of two hydrophobic fatty acid “tails” and a hydrophilic “head” consisting of a phosphate group. The two components are usually joined together by a glycerol molecule. The phosphate groups can be modified with simple organic molecules such as choline, ethanolamine or serine resulting in PC, PE or PS, respectively. PE may be synthesized by the addition of cytidine diphosphate-ethanolamine to diglycerides, releasing cytidine monophosphate. PS is biosynthesized in bacteria by condensing the amino acid serine with CDP (cytidine diphosphate)-activated phosphatidic acid.

As appreciated, production of at least one phospholipid, including, for example, PE, may be via multiple endogenous (i.e. of the microorganism) pathways.

As also described herein, it was suggested that the at least one phospholipid, including, for example, PE, produced by at least one microorganism may be secreted into the host's gut and mediate processes in the host, including, inter alia, endocannabinoid metabolism.

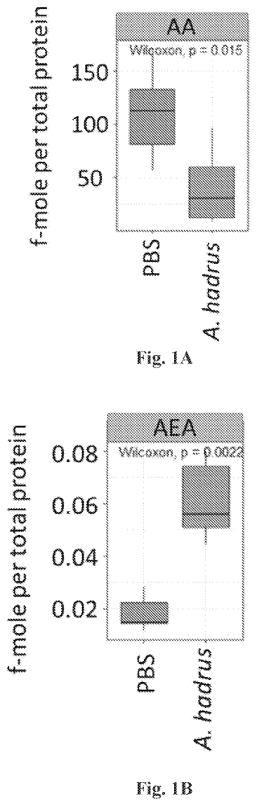

As shown in the examples below, specifically Example 1 and related FIGS. 1A and 1B, treatment of macrophages with Anderostipes hadrus DSM 3319, reduced arachidonic acid levels and increased anandamide synthesis in the macrophages. These results suggest a novel microorganism-host inter-related mechanism in which the at least one phospholipid, such as PE, produced by the microorganisms is secreted to the host (possibly to the host gut) where it serves as a precursor for anandamide synthesis by the host cells. In other words, production of, the at least one phospholipid, including, for example, PE, by microorganisms increased host's anandamide levels.

Anandamide (ANA) (also known as N-arachidonoylethanolamine (AEA)), (denoted herein as ANA or AEA) is a fatty acid neurotransmitter derived from the non-oxidative metabolism of eicosatetraenoic acid (arachidonic acid), an essential omega-6 fatty acid. Anandamide is synthesized by neurons as well as macrophages in a calcium-dependent manner. It is degraded primarily by the fatty acid amide hydrolase (FAAH) enzyme, which converts anandamide into ethanolamine and arachidonic acid. Endocannabinoids, including anandamide and 2-arachidonyl glycerol (2-AG) are lipid mediators that are made on demand and activate cannabinoid and vanilloid receptors to give rise to a variety of cellular responses including physiological and pathophysiological actions controlling motility, secretion, and intestinal inflammation.

Hence, in accordance with some aspects which may be implemented as embodiments of the invention, the microbial consortium comprising two or more microorganisms, at least one of the two or more microorganisms is capable of modulating endocannabinoid metabolism in a host.

In accordance with some other embodiments, the microbial consortium comprising two or more microorganisms, at least one of the two or more microorganisms is capable of modulating anandamide levels in a host.

In accordance with some further embodiments, the microbial consortium comprising two or more microorganisms, at least one of the two or more microorganisms is capable of modulating the production of AEA in a host.

In accordance with some further embodiments, the microbial consortium comprising two or more microorganisms, at least one of the two or more microorganisms is capable of modulating AEA levels in a host.

In accordance with some embodiments, the microbial consortium comprising two or more microorganisms, at least one of the two or more microorganisms is capable of modulating at least one phospholipid, capable of modulating endocannabinoid metabolism in a host or combination thereof.

In accordance with some embodiments, the microbial consortium comprising two or more microorganisms, at least one of the two or more microorganisms is capable of modulating PE, capable of modulating endocannabinoid metabolism in a host or combination thereof.

In accordance with some embodiments, the microbial consortium comprising two or more microorganisms, at least one of the two or more microorganisms is capable of modulating at least one phospholipid, capable of increasing AEA levels in a host or combination thereof.

In accordance with some embodiments, the microbial consortium comprising two or more microorganisms, at least one of the two or more microorganisms is capable of producing PE, capable of increasing AEA levels in a host or combination thereof.

In addition to modulating at least one phospholipid, AEA or a combination of the two, the one or more of the microorganisms in the microbial consortium of the present invention is capable of modulating additional physiological/biological processes, some of which may be related.

In other words, the at least two or more microorganisms in the microbial consortium may possess various biological relationship such that, for example, at least one microorganism may benefit from the at least one other microorganism. For example, a metabolic product of one microorganism of the microbial consortium may be used as a substrate by another microorganism of the microbial consortium or alternatively by additional gut commensals. This together may increase the likelihood of intestinal colonization by the microbial consortium or promoting a desired activity of one or more microorganism.

It was suggested that the combination of at least two microorganisms in the microbial consortium may achieve actions resulting in immune modulation through several underlying, overlapping and complementary mechanisms (processes). For example, at least one microorganism in the microbial consortium may be capable of modulating at least one process, at times at least two processes and even at times at least three or at least four process as detailed herein below.

Additionally or alternatively, a specific process may be modulated by a single microorganism, or at times by at least two microorganisms, at least three microorganisms or even by at least four microorganisms in the microbial consortium.

While some of the modulated processes may overlap between the two or more microorganisms, the consortium has a degree of diversity in order to obtain a broad and complementary effect. For example and as noted herein, the microbial consortium of the invention comprises two or more microorganism that can modulate at least one short chain fatty acid (SCFA), lactate or a combination thereof.

Accordingly, in some embodiments, the two or more microorganisms in the microbial consortium are capable of modulating at least one SCFA, lactate or a combination thereof.

Hence, in accordance with some aspects, the present disclosure provides a microbial consortium comprising two or more microorganisms, the two or more microorganisms are capable of modulating at least one phospholipid and capable of modulating at least one of SCFA, lactate or combination thereof.

In accordance with some embodiments, the two or more microorganisms are capable of modulating PE and modulating one or more of SCFA, lactate or a combination thereof.

The one or more of PE, at least one SCFA or lactate may be secreted from the microorganism to a host, to modulate physiological/biological processes in a host.

A process in a host may be modulated, for example by a microorganism's enzyme.

In some embodiments, the two or more microorganisms in the microbial consortium are capable of modulating host' secondary bile acid, polysaccharide or glycosaminoglycan (GAG). As detailed herein below, it was suggested that secondary bile acid, polysaccharide, GAG or combination thereof in a host may be modulated by an enzyme which is produced by at least one microorganism in the microbial consortium, which is secreted into the host gut.

In some embodiments, the two or more microorganisms in the microbial consortium are capable of modulating one or more of at least one secondary bile acid in a host, a polysaccharide in a host, glycosaminoglycan (GAG) in a host or any combination thereof.

Hence, in accordance with some aspects, the present disclosure provides a microbial consortium comprising two or more microorganisms, the two or more microorganisms are capable of modulating at least one phospholipid and modulating one or more of (i) a secondary bile acid, (ii) a polysaccharide, (iii) a GAG or (iv) a combination thereof.

In accordance with some aspects, the present disclosure provides a microbial consortium comprising two or more microorganisms, the two or more microorganisms are capable of modulating at least one phospholipid and modulating one or more of (i) SCFA, (ii) lactate, (iii) secondary bile acid, (iv) a polysaccharide, (v) a GAG or (vi) any combination thereof.

The term modulation or modulating refers to a change, modification or the like in the specified item and encompasses a change that results in either activation/increase activity or inhibition/decrease activity of a specified item (e.g. lactate), such as a physiological/cellular process and specifically, any of the processes/products of the invention as specified herein.

Hence, the microbial consortium comprising two or more microorganisms may be considered as either an activator or an inhibitor of a specific process.

An activator refers to a microorganism that induce, activate, stimulate, increase, facilitate, enhance activation, sensitize or up regulate at least one physiological/cellular process.

An inhibitor refers to a microorganism that inhibit, partially or totally block stimulation or activation, decrease, prevent, delay activation, inactivate, desensitize, or down regulate a physiological/cellular process.

As described herein, the present invention encompasses process which are modulated endogenous in the microorganisms and processes which are modulated in the host by products modulated in the microorganism and secreted therefrom to a host.

Processes that are modulated endogenous in the microorganisms, i.e. microbial-endogenous processes are all collectively denoted herein at times as “microbial processes”. The microbial processes may take place in at least one specific type (species or microorganism) microorganism (endogenous process). Such microbial processes produce (at times by a specific process of the invention), for example, a microorganism product that may be secreted from the microorganism to affect the host.

Accordingly, the microbial consortium may, for example, modulate (by activation) a microbial process resulting in the production of a product. The product includes, for example, one or more of SCFA, lactate, PE or a combination thereof. As detailed herein, the one or more products may by secreted to the host (possibly host gut).

Additionally, the secreted product may be used by the host (i.e. using host components) for modulating a process in the host.

As described herein, the microorganisms of the invention were found to modulate a variety of cellular processes in a host, such process are collectively denoted herein as “host processes”.

These processes may be mediated by a microbial component, produced by a microorganism and secreted to the host. Thus, at least one of the two or more microorganisms, for example the at least one microorganism that is capable of producing at least one phospholipid is capable of modulating a cellular process in a host subject (functional host process).

Without being bound by theory, it was suggested that an interaction exists between a specific process in the microorganisms and a cellular process being modulated, for example activated, by this specific process (or product of the process) in a host subject.

As described herein, modulation of a physiological process may result in production of a product (e.g. at least one SCFA). As used herein the production/synthesizing a product by at least one microorganism encompasses, at times, the secretion of the product from the at least one microorganism into the host's gut and stool.

In accordance with some embodiments, the microbial consortium comprises two or more microorganisms, the two or more microorganisms are capable of producing at least one phospholipid and producing at least one of (i) at least one SCFA and (ii) lactate.

In accordance with some other embodiments, the microbial consortium comprises two or more microorganisms, the two or more microorganisms are capable of modulating the production of at least one endocannabinoid and having one or more of the following features (i) producing a secondary bile acid, (ii) degrading a polysaccharide, (iii) degrading a GAG or (iv) any combination thereof.

In accordance with some other embodiments, the microbial consortium comprises two or more microorganisms, the two or more microorganisms are capable of modulating the production of at least one phospholipid, an endocannabinoid or any combination thereof and having one or more of the features (i) producing a secondary bile acid, (ii) degrading a polysaccharide, (iii) degrading a GAG or (iv) any combination thereof.

In accordance with some further embodiments, the present disclosure provides a microbial consortium comprising two or more microorganisms, the two or more microorganisms are capable of producing at least one phospholipid, modulate endocannabinoid production or any combination thereof and having one or more of the following (i) producing at least SCFA, (ii) producing lactate, (iii) producing a secondary bile acid, (iv) degrading a polysaccharide, (v) degrading a GAG or (vi) any combination thereof.

It should be noted that the at least one microorganism being capable of producing a phospholipid, modulate endocannabinoid production or a combination thereof may also be capable of at least one of (i) producing at least one SCFA, (ii) producing lactate, (iii) producing secondary bile acid, (iv) degrading a polysaccharide and (v) degrading a GAG.

It should be further noted that the at least one microorganism being capable of producing a phospholipid, modulate endocannabinoid production or a combination thereof may also be as the at least one other (different) microorganism.

In accordance with some embodiments, at least one of the two or more microorganisms in the microbial consortium is capable of modulating at least one phospholipid, an endocannabinoid or any combination thereof and at least one other (i.e. a different microorganism) of the two or more microorganisms is characterized by having one or more of (i) producing at least one SCFA, (ii) producing lactate, (iii) producing secondary bile acid, (iv) degrading a polysaccharide, (v) degrading a GAG or (vi) any combination thereof.

In accordance with some embodiments, at least one of the two or more microorganisms in the microbial consortium is capable of modulating at least one phospholipid, an endocannabinoid or any combination thereof and at least one other (i.e. a different microorganism) of the two or more microorganisms is characterized by having one or more of (i) producing at least one SCFA, (ii) producing lactate, (iii) producing secondary bile acid, or (iv) any combination thereof.

In accordance with some embodiments, at least one of the two or more microorganisms in the microbial consortium is capable of modulating at least one phospholipid, an endocannabinoid or any combination thereof and at least one other (i.e. a different microorganism) of the two or more microorganisms is characterized by having one or more of (i) producing at least one SCFA, (ii) producing secondary bile acid, (iii) degrading a polysaccharide, (iv) degrading a GAG or (v) any combination thereof.

In accordance with some embodiments, at least one of the two or more microorganisms in the microbial consortium is capable of modulating at least one phospholipid, an endocannabinoid or any combination thereof and at least one other of the two or more microorganisms is characterized by having one or more of (i) producing at least one SCFA, (ii) producing secondary bile acid, (iii) degrading a polysaccharide, (iv) producing lactate or (v) any combination thereof.

In accordance with some embodiments, at least one of the two or more microorganisms in the microbial consortium is capable of modulating at least one phospholipid, an endocannabinoid or any combination thereof and at least one other of the two or more microorganisms is characterized by having one or more of (i) producing at least one SCFA, (ii) producing secondary bile acid, (iii) producing lactate or (iv) any combination thereof.

In accordance with some embodiments, at least one of the two or more microorganisms in the microbial consortium is capable of modulating at least one phospholipid, an endocannabinoid or any combination thereof and at least one other of the two or more microorganisms is characterized by having one or more of (i) producing at least one SCFA, (ii) producing secondary bile acid, or (iii) any combination thereof.

In some embodiments, at least one of the two or more microorganisms is characterized by producing SCFA and producing secondary bile acid.

In some embodiments, at least one of the two or more microorganisms is characterized by producing SCFA, degrading a polysaccharide, degrading a GAG or combination thereof.

In some embodiments, at least one of the two or more microorganisms is characterized by producing SCFA, producing lactate, degrading a polysaccharide or combination thereof.

In some embodiments, at least one of the two or more microorganisms is characterized by producing SCFA, producing lactate or combination thereof.

In some embodiments, at least one of the two or more microorganisms is characterized by producing lactate.

In some embodiments, at least one of the two or more microorganisms is characterized by producing SCFA.

In some embodiments, two of the two or more microorganisms are capable of producing SCFA.

In some embodiments, at least one of the two or more microorganisms is characterized by producing a phospholipid, an endocannabinoid or a combination thereof and at least one SCFA.

In some embodiments, at least one of the two or more microorganisms is characterized by producing a phospholipid and at least one SCFA.

In some embodiments, the at least one phospholipid is PE, PC, PS and any combination thereof.

In some embodiments, the at least one phospholipid is PE.

In some embodiments, at least one of the two or more microorganisms is characterized by producing PE and at least one SCFA.

In some embodiments, at least one of the two or more microorganisms is characterized by modulating production of an endocannabinoid and at least one SCFA.

In some embodiments, at least one of the two or more microorganisms is characterized by modulating production of AEA and producing at least one SCFA.

The processes modulated by the identified microorganism include also modulation of SCFA. It was suggested that the at least one SCFA typically produced by the gut microbiome, is augmented by the microbial consortium of the invention. The produced SCFA may be secreted to the host's gut.

In some embodiments, the at least one SCFA comprises at least one of an acetic acid, a propionic acid, a butyric acid, a valeric acid, an isovaleric acid, a formic acid, an isobutyric acid or any combination thereof.

In some other embodiments, the at least one SCFA comprises at least one of an acetic acid, a propionic acid, a butyric acid or any combination thereof.

In some embodiments, at least one of the two or more microorganisms is capable of producing at least one of acetic acid, butyric acid, propionic acid or any combination thereof.

In some embodiments, at least one of the two or more microorganisms is characterized by producing acetic acid and producing secondary bile acid.

In some embodiments, at least one of the two or more microorganisms is characterized by producing butyric acid, degrading a polysaccharide and degrading a GAG.

In some embodiments, at least one of the two or more microorganisms is characterized by producing acetic acid, producing propionic acid, producing lactate and degrading a polysaccharide.

In some embodiments, at least one of the two or more microorganisms is characterized by producing acetic acid and producing lactate.

In some embodiments, at least one of the two or more microorganisms is characterized by producing lactate.

In some other embodiments, at least one of the two or more microorganisms is capable of producing a phospholipid, an endocannabinoid or any combination thereof and at least one of the two or more microorganisms in the microbial consortium is capable of producing at least one of acetic acid, butyric acid and propionic acid.

In some embodiments, at least one of the two or more microorganisms is capable of producing PE and butyric acid. In some embodiments, at least one of the two or more microorganisms is capable of producing PE and acetic acid. In some embodiments, at least one of the two or more microorganisms is capable of producing PE and propionic acid.

In some embodiments, at least one of the two or more microorganisms is capable of modulating the production of AEA and butyric acid. In some embodiments, at least one of the two or more microorganisms is capable of modulating production of AEA and capable of producing acetic acid. In some embodiments, at least one of the two or more microorganisms is capable of modulating production of AEA and propionic acid.

In some embodiments, at least one of the two or more microorganisms in the microbial consortium is capable of producing lactate.

When referring to lactate modulation by the at least one of the two or more microorganisms in the microbial consortium it should be understood as production of lactate by the microorganisms and optionally its secretion to the host's gut.

In some embodiments, the microbial consortium comprises at least one microorganism that is capable of producing an acetic acid, a propionic acid, lactate or combination thereof.

In some other embodiments, at least one of the two or more microorganisms is capable of producing a PE and at least one microorganism that is capable of producing an acetic acid, a propionic acid, lactate or any combination thereof.

In some other embodiments, at least one of the two or more microorganisms is capable of modulating production of AEA and at least one microorganism that is capable of producing an acetic acid, a propionic acid, lactate or any combination thereof.

In some embodiments, the microbial consortium comprises at least one microorganism that is capable of producing acetic acid and lactate.

In some other embodiments, at least one of the two or more microorganisms is capable of producing a PE and at least one microorganism that is capable of producing acetic acid and lactate.

In some other embodiments, at least one of the two or more microorganisms is capable of modulating production of AEA and at least one microorganism that is capable of producing acetic acid and lactate.

In some embodiments, the identified microorganisms (forming the microbial consortiums of the invention) modulate secondary bile acid production. Bile acid are amphipathic steroid molecules found in the bile of mammals and other vertebrates.

In accordance with the present disclosure, those microorganism(s) that modulate secondary bile acid are in fact microorganism(s) that are capable of transforming/converting host's primary bile acids to secondary bile acids. Hence, the secondary bile acid in the context of the invention refers to a host product following modification by microorganism's activity, specifically those microorganisms in the microbial consortium

Conversion of host's primary bile acids to secondary bile acids may be done by any microorganisms' enzyme capable of this reaction. For example, an enzyme may include, inter alia, 7-alpha-dehydroxylase.

In some embodiments, at least one of the two or more microorganisms in the microbial consortium modulates secondary bile acid. As noted above, the two or more microorganisms are affecting the synthesis of secondary bile acids in the host gut hence modulating the secondary bile acids level (content/amount) in a host.

In accordance with some other embodiments, at least one of the two or more microorganisms in the microbial consortium is capable of producing secondary bile acid in the host.

In some embodiments, the secondary bile acid comprises deoxycholic acid (DOC), lithocholic acid (LCA) or any combination thereof.

In some embodiments, at least one of the two or more microorganisms in the microbial consortium is capable of producing DOC.

In some embodiments, at least one of the two or more microorganisms in the microbial consortium is capable of producing LCA.

DOC is formed from cholic acid, whereas LCA is formed from chenodeoxycholic acid.

Additionally, the microbial consortium is capable of modulating polysaccharides.

A polysaccharide is a polymeric carbohydrate molecule composed of long chains of monosaccharide units bound together by glycosidic linkages, and on hydrolysis give the constituent monosaccharides or oligosaccharides.

The microbial consortium is capable of modulating polysaccharides, by degrading them for example by using specific enzymes from microorganisms. Hence, when referring to modulation of polysaccharide it should be understood as host polysaccharides (host substrate) and host dietary polysaccharides being modulated (degraded) by a microorganisms' component. Such microorganisms' component is in accordance with some embodiments, an enzyme. Non-limiting examples of enzymes include pectate lyase, rhamnogalacturonate lyase, glucuronyl hydrolase, alginate lyase or cellulase.

In some embodiments, at least one of the two or more microorganisms in the microbial consortium modulates polysaccharides by their degradation.

In some embodiments, at least one of the two or more microorganisms in the microbial consortium degrades at least one polysaccharide. In some embodiments, the at least one of the two or more microorganisms in the microbial consortium degrades at least one of a plant cell wall polysaccharide. Non-limiting examples of plant cell wall polysaccharide include starch, pectin, inulin, alginate, mucin or fructan.

Still further, the microbial consortium is capable of modulating at least one GAG.

When referring to modulation of GAG it should be understood as host's GAG being modulated (degraded) by a microorganisms' component, such as GAG-degrading enzymes, i.e. enzymes that cleave (depolymerize) glycosaminoglycans (GAGs).

GAGs (also denoted as mucopolysaccharides) are long linear (unbranched) polysaccharides consisting of repeating disaccharide (double sugar) units. GAGs include, for example, heparin/heparan sulfate (HSGAGs) and chondroitin sulfate/dermatan sulfate (CSGAGs).

In some embodiments, at least one of the two or more microorganisms in the microbial consortium is capable of modulating GAG by producing at least one enzyme that cleave (depolymerize) host's GAGs such as HSGAGs and/or CSGAGs.

In some embodiments, the at least one of the two or more microorganisms includes at least one enzyme being a heparinase, chondroitin lyase or any combination thereof.

Heparinase and chondroitin lyase are enzymes found in at least one of the two or more microorganisms in the microbial consortium, that are capable of degrading host's GAG. Heparinase is an enzyme that catalyses the hydrolytic cleavage of heparins or heparan sulfate motifs on complexed glucosaminoglycans.

In some embodiment, at least one of the two or more microorganisms in the microbial consortium is capable of producing heparinase.

In some other embodiments, at least one of the two or more microorganisms in the microbial consortium is capable of producing chondroitin lyase.

Modulation of host' GAG is in accordance with some embodiments, via chondroitin lyase from at least one of the two or more microorganisms.

In accordance with some embodiments, the microbial consortium is characterized by having one or more of (i) producing a phospholipid, specifically PE, (ii) modulating production of endocannabinoid, specifically AEA, (iii) producing lactate, (iv) producing acetic acid, (iv) producing butyric acid, (v) producing propionic acid, (vi) producing at least one bile acid, specifically DOC, LCA or any combination thereof, (vii) degrading at least one polysaccharide, (viii) degrading at least one GAG or (ix) any combination thereof.

In accordance with some embodiments, at least one microorganism in the microbial consortium is characterized by having one or more of (i) producing a phospholipid, specifically PE, (ii) modulating production of an endocannabinoid, specifically AEA or (iii) any combination thereof and at least one other microorganism in the microbial consortium is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

As described herein, the microbial consortium is capable of modulating various cellular process in a subject (for example in a host subject).

Without being bound by theory, the inventors suggested that there may be a connection between the endogenous modulated process and the host's cellular process. For example, it was suggested that production of phospholipid and/or production of SCFA and/or production of secondary bile acids may affect the differentiation of specific immune cell population and/or increase the secretion of specific mediators from immune cells or affect gut mucosal cells in a host subject.

Thus, in some embodiments, at least one of the two or more microorganisms is capable of modulating at least one cellular process in a host subject, such processes are collectively denoted herein as “host processes”. As used herein the term “host processes” refers to physiological/biological/chemical process in the host that is modulated (activated or inhibited as defined herein above) via a product produced by the microorganisms, including, inter alia, at least one phospholipid, lactate, or any combination thereof.

In some embodiments, the microbial consortium is capable of modulating one or more of (i) Regulatory T cells (Tregs), (ii) an anti-inflammatory cytokine, (iii) a bile acid receptor (iv) gut barrier integrity, (v) nuclear factor kappa-light-chain-enhancer of activated B cells (NF-κB), (vi) an inflammasome, (vii) a pro-inflammatory cytokine or (viii) any combination thereof.

The term “capable of modulating” as previously defined herein, encompasses any change (positive or negative) in the level/amount/concentration/content and any related expression as defined herein.

For example, modulating a process by activation may contribute to promoting an anti-inflammatory effect and/or gut health whereas modulating a process by inhibition may contribute to decreasing pro-inflammatory effect. The modulation collectively may ultimately affect the immune system and/or the gastrointestinal system.

In some embodiments, the microbial consortium increase/induce at least one cellular process in a host.

In some embodiments, the microbial consortium is capable of activating processes that contribute to an anti-inflammatory effect and/or gut health.

In some embodiments, the microbial consortium is capable of having one or more of (i) activating Tregs, (ii) activating an anti-inflammatory cytokine, (iii) activating a bile acid receptor (iv) activating gut barrier integrity, (v) inhibiting NF-κB, (vi) inhibiting an inflammasome, (vii) inhibiting a pro-inflammatory cytokine or (viii) any combination thereof.

In some embodiments, the microbial consortium is capable of activating one or more (i) Tregs, (ii) an anti-inflammatory cytokine, (iii) a bile acid receptor, (iv) gut barrier integrity or (v) any combination thereof.

In some embodiments, the microbial consortium is capable of activating (inducing) Tregs. It was suggested that the microbial consortium increased the amount/number of Tregs. Tregs are a subpopulation of T cells that modulate the immune system, maintain tolerance to self-antigens, and prevent autoimmune disease.

In some embodiments, the microbial consortium is capable of inducing production (synthesis) of at least one anti-inflammatory cytokine.

In some embodiments, the at least one anti-inflammatory cytokine is at least one of IL-4, IL-10, IL-11, IL-13 or any combination thereof.

It is suggested that the microbial consortium may increase the amount/level of at least one anti-inflammatory cytokine in the host.

In some embodiments, the microbial consortium is capable of inhibiting an pro-inflammatory cytokine.

Pro-inflammatory cytokine is type of cytokine that is secreted from immune cells and certain other cell types that promotes inflammation.

In some embodiments, the pro-inflammatory cytokine is at least one of interleukin-1 (IL-1), IL-2, IL-6, IL-12, IL-17, IL-22, IL-23 and IL-18, TNF-α, interferon gamma (IFNγ), granulocyte-macrophage colony stimulating factor (GM-CSF) or any combination thereof.

In some embodiments, the microbial consortium is capable of inhibiting secretion of TNF□. TNFD□□ is a cell signaling protein (cytokine) involved in systemic inflammation and is one of the cytokines that make up the acute phase reaction. The primary role of TNF is in the regulation of immune cells.

In some embodiments, the microbial consortium is capable of activating a bile acid receptor.

It was suggested that the microbial consortium modulate secondary bile acid production by comprising increased presence of microorganisms that are capable of modulating this function. For example, the microbial consortium has increased microorganism population that comprises at least one enzyme which is capable of transforming host's primary bile acids to secondary bile acids.

Thus, it was further suggested that the activation of a bile acid receptor is due to increased amounts of secondary bile acids produced by the microorganisms of the microbial consortium of the invention.

The bile acid receptor may be any receptor identified in the art. In some embodiments, the bile acid receptor is farnesoid X receptor (FXR), G protein-coupled bile acid receptor 1 (GPBAR1) or a combination thereof.

In some embodiment, the bile acid receptor is FXR. FXR also known as NR1H4 (nuclear receptor subfamily 1, group H, member 4) is a nuclear receptor that is encoded by the NR1H4 gene in humans.

In some embodiment, the bile acid receptor is GPBAR1. GPBAR1 also known G-protein coupled receptor 19 (GPCR19), membrane-type receptor for bile acids (M-BAR) or TGR5 as is a protein that in humans is encoded by the GPBAR1 gene.

In some embodiments, the microbial consortium is capable of increasing gut barrier integrity. The term “gut barrier integrity” encompasses upregulation of cellular adhesion molecules (proteins). The cellular adhesion proteins are located on the cell surface and are involved in binding with other cells or with the extracellular matrix (ECM) in the process called cell adhesion. Non-liming examples of cellular adhesion proteins include tight-junctions connecting adjacent cells and components within the tight junctions and in colon mucins, desomsomes and hemidesmosomes.

In some embodiments, the microbial consortium is capable of inhibition/reduction of a cellular process.

In some embodiments, the microbial consortium is capable of inhibiting/reducing pro-inflammatory processes.

In some embodiments, the microbial consortium is capable of inhibiting/reducing (i) NF-κB, (ii) an inflammasome, (iii) a pro-inflammatory cytokine.

In some embodiments, the microbial consortium is capable of inhibiting an inflammasome.

In some embodiments, the microbial consortium is capable of inhibiting/reducing NF-κB.

An inflammasome is a multiprotein oligomer responsible for the activation of inflammatory responses and promotes the maturation and secretion of pro-inflammatory cytokines, such as Interleukin 1β (IL-1β) and Interleukin18 (IL-18) as well as additional interleukins).

Thus, collectively, the at least two microorganisms in the microbial consortium may influence host tissue and modulate the immune system. Exemplary mechanisms include secondary bile acids production/synthesis, production of SCFA, increased polysaccharides utilization and induction of Tregs.

It was suggested that the microbial consortium is capable of increasing in IL-10 secretion, induction of Tregs, reducing NF-kB activation, reducing IL-1 secretion, reducing IL-6 secretion, reducing inflammasome activation, combatting reactive oxygen species or any combination thereof.

In addition, the at least two microorganisms in the microbial consortium may promote the maintenance of gut barrier integrity.

In some embodiments, the microbial consortium is capable of having one or more of (i) producing at least one phospholipid, specifically PE, (ii) producing at least one SCFA, (iii) producing lactate, (iv) producing secondary bile acid, (v) degrading at least one polysaccharide, (vi) degrading at least one GAG, (vii) activating Tregs, (viii) activating an anti-inflammatory cytokine, (ix) activating a bile acid receptor (x) activating gut barrier integrity, (xi) inhibiting NF-κB, (xii) inhibiting an inflammasome, (xiii) inhibiting a pro-inflammatory cytokine or (xiv) any combination thereof.

In some embodiments, at least one of the two or more microorganisms is characterized by one or more of (i) producing at least one SCFA, specifically acetic acid, (ii) producing secondary bile acid, (iii) activating Tregs, (iv) producing IL-10, (v) activating a bile acid receptor, (vi) activating gut barrier integrity, (vii) inhibiting TNF-a, (viii) inhibiting an inflammasome, (ix) inhibiting NF-κB, (x) inhibiting a pro-inflammatory cytokine or (xi) any combinations thereof.

In some embodiments, at least one of the two or more microorganisms is characterized by one or more of (i) producing at least one SCFA, specifically butyric acid, (ii) producing at least one phospholipid, specifically PE (iii) activating Tregs, (iv) producing IL-10, (v) activating gut barrier integrity, (vi) inhibiting TNF-a, (vii) inhibiting NF-κB, (viii) inhibiting a pro-inflammatory cytokine or (ix) any combinations thereof.

In some embodiments, at least one of the two or more microorganisms is characterized by (i) producing at least one SCFA, specifically butyric acid (ii) degrading at least one polysaccharide, (iii) degrading at least one GAG, (iv) activating Tregs, (v) producing IL-10, (vi) activating gut barrier integrity, (vii) inhibiting TNF-a (viii) inhibiting NF-κB, (ix) inhibiting a pro-inflammatory cytokine or (x) any combination thereof.

In some embodiments, at least one of the two or more microorganisms is characterized by (i) producing at least one SCFA, specifically acetic acid, propionic acid or a combination thereof (ii) producing lactate, (iii) degrading at least one polysaccharide, (iv) activating Tregs, (v) producing IL-10, (vi) inhibiting TNF-a (vii) inhibiting NF-κB, (viii) inhibiting a pro-inflammatory cytokine or (ix) any combination thereof.

In some embodiments, at least one of the two or more microorganisms is characterized by (i) producing at least one SCFA, specifically acetic acid (ii) producing lactate, (iii) activating Tregs, (iv) producing IL-10, (v) activating gut barrier integrity (vi) inhibiting TNF-a (vii) inhibiting NF-κB, (viii) inhibiting a pro-inflammatory cytokine or (ix) any combination thereof.

In some embodiments, at least one of the two or more microorganisms is characterized by (i) producing lactate, (iii) activating Tregs, (iii) producing IL-10 or (iv) any combination thereof.

As described herein, the microbial consortium comprises at least two microorganisms, each as defined herein or any combination of each one of the microorganisms described herein.

When referring to a microbial consortium comprising at least two microorganisms it should be understood as referring to two different microorganisms (i.e. different strains). The two different microorganisms may be within the same microorganism genus or within the microorganism species.

For examples, the two or more microorganisms can belong both to one genus such as Anaerostipes genus but to different species within or alternatively to the same genus, the same species but different strains. Alternatively, the two or more microorganisms can belong each to a different genus.

In some embodiments, the microbial consortium comprises at least one microorganism from the Anaerostipes genus.

In some embodiments, the microbial consortium comprises two or more microorganisms belonging to one or more of the Clostridium genus, Anaerostipes genus, Bacteroides genus, Phascolarctobacterium genus, Megamonas genus, Lactococcus genus, Paeniclostridium genus, Parabacteroides genus, Eubacterium genus, Lachnospiraceae genus, Ruminococcus genus, Proteocatella genus, Faecalibacterium genus, Roseburia genus, Butyricicoccus genus, Lactobacillus genus, Leuconostoc genus, Streptococcus genus, Oenococcus genus, Bifidobacterium genus or combinations thereof.

In some embodiments, the microbial consortium comprises two or more microorganisms belonging to one or more of the Clostridium genus, Anaerostipes genus, Bacteroides genus, Megamonas genus, Lactococcus genus, or combinations thereof.

In some embodiments, the microbial consortium comprises at least one microorganism from the Clostridium genus. Clostridium genus may be denoted by Taxonomy ID (or taxid): 1485. Clostridium is a genus of Gram-positive bacteria.

In some embodiments, the microbial consortium comprises at least one microorganism from the Anaerostipes genus. Anaerostipes genus may be denoted by Taxonomy ID: 207244. Anaerostipes is a genus of Gram positive and anaerobic bacterial.

In some embodiments, the microbial consortium comprises at least one microorganism from the Bacteroides genus. Bacteroides genus may be denoted by Taxonomy ID: 816. Bacteroides is a genus of Gram-negative, obligate anaerobic bacteria.

In some embodiments, the microbial consortium comprises at least one microorganism from the Phascolarctobacterium genus. Phascolarctobacterium genus may be denoted by Taxonomy ID: 33024. Phascolarctobacterium is a genus of can produce short-chain fatty acids, including acetate and propionate, and can be associated with the metabolic state and mood of the host.

In some embodiments, the microbial consortium comprises at least one microorganism from the Megamonas genus. Megamonas genus may be denoted by Taxonomy ID: 158846.

In some embodiments, the microbial consortium comprises at least one microorganism from the Lactococcus genus. Lactococcus genus may be denoted by Taxonomy ID: 1357. Lactococcus is a genus of lactic acid bacteria.

In some embodiments, the microbial consortium comprises at least one microorganism from the Parabacteroides genus. Parabacteroides genus may be denoted by Taxonomy ID (or taxid): 375288. Parabacteroides is a Gram-negative, anaerobic, non-spore-forming genus.

In some embodiments, the microbial consortium comprises at least one microorganism from the Eubacterium genus. Eubacterium genus may be denoted by Taxonomy ID (or taxid): 1730. Eubacterium is a genus of Gram-positive bacteria and are characterized by a rigid cell wall.

In some embodiments, the microbial consortium comprises at least one microorganism from the Lachnospiraceae genus. Lachnospiraceae genus may be denoted by Taxonomy ID (or taxid): 186803. Lachnospiraceae is a genus of are a family of anaerobic, spore-forming bacteria.

In some embodiments, the microbial consortium comprises at least one microorganism from the Proteocatella genus. Proteocatella genus may be denoted by Taxonomy ID (or taxid): 181069.

In some embodiments, the microbial consortium comprises at least one microorganism from the Ruminococcus genus. Ruminococcus genus may be denoted by Taxonomy ID (or taxid): 1263.

In some embodiments, the microbial consortium comprises at least one microorganism from the Faecalibacterium genus. Faecalibacterium genus may be denoted by Taxonomy ID (or taxid): 216851.

In some embodiments, the microbial consortium comprises at least one microorganism from the Roseburia genus. Roseburia genus may be denoted by Taxonomy ID (or taxid): 841.

In some embodiments, the microbial consortium comprises at least one microorganism from the Butyricicoccus genus. Butyricicoccus genus may be denoted by Taxonomy ID (or taxid): 580596.

In some embodiments, the microbial consortium comprises at least one microorganism from the Lactobacillus genus. Lactobacillus genus may be denoted by Taxonomy ID (or taxid): 1578.

In some embodiments, the microbial consortium comprises at least one microorganism from the Leuconostoc genus. Leuconostoc genus may be denoted by Taxonomy ID (or taxid): 1243.

In some embodiments, the microbial consortium comprises at least one microorganism from the Streptococcus genus. Streptococcus genus may be denoted by Taxonomy ID (or taxid): 1301.

In some embodiments, the microbial consortium comprises at least one microorganism from the Oenococcus genus. Oenococcus genus may be denoted by Taxonomy ID (or taxid): 46254.

In some embodiments, the microbial consortium comprises at least one microorganism from the Bifidobacterium genus. Bifidobacterium genus may be denoted by Taxonomy ID (or taxid): 1678.

In some embodiments, the microbial consortium comprises at least one microorganism from the Anaerostipes hadrus species.

In some embodiments, the microbial consortium comprises at least two microorganisms species belonging to species selected from Clostridium hiranonis species, Anaerostipes hadrus species, Bacteroides stercoris species, Megamonas hypermegale species, Clostridium bolteae species, Lactococcus lactis species, Phascolarctobacterium succinatutens species, Clostridium scindens species, Clostridium hylemonae species, Parabacteroides distasonis species, Eubacterium limosum species, Clostridium leptum species, Proteocatella sphenisci species, Lachnospiraceae 5_1_57FAA species, Eubacterium rectale species, Clostridium sp. SS2/1 species, Lachnospiraceae bacterium species, Eubacterium hallii species, Clostridium clostridioforme species, Anaerostipes caccae species, Ruminococcus albus species, Clostridium cellulovorans species, Faecalibacterium prausnitzii species, Clostridium leptum species, Eubacterium rectale species, Roseburia faecis species, Roseburia imulinivorans species, Roseburia intestinalis species, Roseburia hominis species, Eubacterium hallii species, Anaerostipes butyraticus species, Anaerostipes caccae species, Butyricicoccus pullicaecorum species, Lactobacillus johnsonii species, Lactobacillus plantarum species, Lactobacillus reuteri species, Lactobacillus rhamnosus GG species, Lactobacillus acidophilus species, Lactobacillus gallinarumspecies, Lactobacillus casei species, Lactobacillus paracasei species, Leuconostoc mesenteroides species, Streptococcus thermophiles species, Oenococcus oeni species, Bacteroides thetaiotaomicron species, Bacteroides xylanisolvens species, Bacteroides vulgatus species, Bacteroides fragilis species, Roseburia intestinalis species, Ruminococcus champanellensis species, Bifidobacterium adolescentis species, Pedobacter heparimis species, Bacteroides eggerthii species, Bacteroides thetaiotaomicron species, Lactobacillus rhamnosus species, Lactobacillus animalisspecies, Lactobacillus casei species, Enterococcus faecalis species, Alistipes shahiispecies, Bacteroides thetaiotaomicron species, Bacteroides ovatus species, Bacteroides caccae species subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, Clostridium boltede species, Lactococcus lactis species, Phascolarctobacterium succinatutens species, Clostridium scindens species, Clostridium hylemonde species, Parabacteroides distasonis species, Eubacterium limosum species, Clostridium leptum species, Proteocatella sphenisci species, Lachnospiraceae 5_1_57FAA species, Eubacterium rectale species, Clostridium sp. SS2/1 species, Lachnospiraceae bacterium species, Eubacterium hallii species, Clostridium clostridioforme species, Anaerostipes caccae species, Ruminococcus albus species, Clostridium cellulovorans species, subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, Clostridium bolteae species, Lactococcus lactis species, Phascolarctobacterium succinatutens species, subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, Clostridium bolteae species, Lactococcus lactis species, subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, subspecies or combination thereof.

In some embodiments, the microbial consortium comprises at least two microorganisms species belonging to species selected from Clostridium hiranonis species, Anaerostipes hadrus species, Clostridium bolteae species, Lactococcus lactis species, subspecies or combination thereof.

In some embodiments, the microbial consortium comprises at least two microorganisms species belonging to species selected from Clostridium hiranonis species, Anaerostipes hadrus species, Bacteroides stercoris species and Phascolarctobacterium succinatutens species, subspecies or combination thereof.

In some embodiments, the microbial consortium comprises at least two microorganisms species belonging to species selected from Clostridium hiranonis species, Anaerostipes hadrus species, Clostridium bolteae species, Megamonas hypermegale species, subspecies or combination thereof.

In some embodiments, the microbial consortium comprises at least two microorganisms species belonging to species selected from Clostridium hiranonis species, Anaerostipes hadrus species, Lactococcus lactis species and or subspecies or combination thereof.

In some embodiments, the microbial consortium comprises at least two microorganisms from the Clostridium hiranonis species, Anaerostipes hadrus species or subspecies.

In some embodiments, the microbial consortium comprises at least two microorganisms species belonging to species selected from Clostridium hiranonis species, Anaerostipes hadrus species and Bacteroides stercoris species, subspecies or combination thereof.

In some embodiments, the microbial consortium comprises at least two microorganisms species belonging to species selected from Clostridium hiranonis species, Anaerostipes hadrus species, Megamonas hypermegale species, subspecies or combination thereof.

In some embodiments, the microbial consortium comprises at least two microorganisms species belonging to species selected from Clostridium hiranonis species, Anaerostipes hadrus species, Clostridium bolteae species, subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, Clostridium bolteae species, subspecies or combination thereof.

In some embodiments, the microbial consortium comprises at least two microorganisms species belonging to species selected from Clostridium hiranonis species, Anaerostipes hadrus species, Megamonas hypermegale species, Clostridium boltede species, Lactococcus lactis species, subspecies or combination thereof.

In some embodiments, the microbial consortium comprises at least two microorganisms species belonging to species selected from Clostridium hiranonis species, Anaerostipes hadrus species, Bacteroides stercoris species, Clostridium bolteae species, Lactococcus lactis species, subspecies or combination thereof.

In some embodiments, the microbial consortium comprises at least two microorganisms species belonging to species selected from Clostridium hiranonis species, Anaerostipes hadrus species, Bacteroides stercoris species, Megamonas hypermegale species, Lactococcus lactis species, subspecies or combination thereof.

In some embodiments, the microbial consortium comprises at least two microorganisms species belonging to species selected from Clostridium hiranonis species, Clostridium sp. SS2/1 species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Clostridium sp. SS2/1 species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Lachnospiraceae bacterium species, Bacteroides stercoris species, Phascolarctobacterium succinatutens species, Clostridium bolteae species, Megamonas hypermegale species, and Lactococcus lactis species, subspecies or combination thereof.

in some embodiments, the microbial consortium comprises at least two microorganisms species belonging to species selected from Clostridium hiranonis species, Lachnospiraceae bacterium species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Eubacterium rectale species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Eubacterium rectale species, Bacteroides stercoris species, Clostridium boltede species, Megamonas hypermegale species, and Lactococcus lactis species, subspecies or combination thereof.

In some embodiments, the microbial consortium comprises at least two microorganisms species belonging to species selected from Clostridium hiranonis species, Eubacterium hallii species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Eubacterium hallii species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Clostridium clostridioforme species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Clostridium clostridioforme species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Anaerostipes caccae species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Anaerostipes caccae species, Bacteroides stercoris species, Clostridium boltede species, Megamonas hypermegale species, and Lactococcus lactis species, subspecies or combination thereof.

In some embodiments, the microbial consortium comprises at least two microorganisms species belonging to species selected from Clostridium hiranonis species, Ruminococcus albus species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Clostridium cellulovorans species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Ruminococcus albus species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Clostridium hiranonis species, Clostridium cellulovorans species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Parabacteroides distasonis species, Anaerostipes hadrus species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Parabacteroides distasonis species, Anaerostipes hadrus species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Eubacterium limosum species, Anaerostipes hadrus species, Bacteroides stercoris species, Phascolarctobacterium succinatutens species, Clostridium bolteae species, Megamonas hypermegale species, and Lactococcus lactis species, subspecies or combination thereof.

In some embodiments, the microbial consortium comprises at least two microorganisms species belonging to species selected from Eubacterium limosum species, Anaerostipes hadrus species, Bacteroides stercoris species, Clostridium bolteae species, Megamonas hypermegale species, and Lactococcus lactis species, subspecies or combination thereof.

In some embodiments, the microbial consortium comprises at least two microorganisms species belonging to species selected from Clostridium scindens species, Anaerostipes hadrus species, Bacteroides stercoris species, Clostridium bolteae species, Megamonas hypermegale species, and Lactococcus lactis species, subspecies or combination thereof.

In some embodiments, the microbial consortium comprises at least two different microorganisms from the Clostridium scindens species, Anaerostipes hadrus species, Bacteroides stercoris species and Megamonas hypermegale species.

In some embodiments, the microbial consortium comprises at least two different microorganisms from the Parabacteroides distasonis species, Anaerostipes hadrus species, Bacteroides stercoris species and Megamonas hypermegale species.

In some embodiments, the microbial consortium comprises at least two different microorganisms from the Eubacterium limosum species, Anaerostipes hadrus species, Bacteroides stercoris species and Megamonas hypermegale species.

In some embodiments, the microbial consortium comprises at least two different microorganisms from the Clostridium scindens species, Anaerostipes hadrus species, Clostridium bolteae species and Lactococcus lactis species or subspecies.

In some embodiments, the microbial consortium comprises at least two different microorganisms from the Parabacteroides distasonis species, Anaerostipes hadrus species, Clostridium bolteae species and Lactococcus lactis species or subspecies.

In some embodiments, the microbial consortium comprises at least two different microorganisms from the Eubacterium limosum species, Anaerostipes hadrus species, Clostridium bolteae species and Lactococcus lactis species or subspecies.

In some embodiments, the microbial consortium comprises at least one microorganism from the Clostridium hiranonis species, Clostridium scindens species, Clostridium hylemonae species, Clostridium bifermentas species, Clostridium leptum species, Parabacteroides distasonis species, Eubacterium limosum species, Proteocatella sphenisci species, Lachnospiraceae bacterium species.

In some embodiments, the microbial consortium comprises at least one microorganism from the Anaerostipes hadrus species, Eubacterium rectale species, Eubacterium hallii species, Clostridium sp. SS2/1 species, Clostridium bolteae species, Clostridium clostridioforme species, Clostridium cellulovorans species, Lachnospiraceae bacterium species, Anaerostipes caccae species, Ruminococcus albus species.

In some embodiments, the microbial consortium comprises at least one microorganism from the Faecalibacterium prausnitzii species, Clostridium leptum species, Eubacterium rectale species, Roseburia faecis species, Roseburia imulinivorans species, Roseburia intestinalis species, Roseburia hominis species, Eubacterium hallii species, Anaerostipes butyraticus species, Anaerostipes caccae species and Butyricicoccus pullicaecorum species.

In some embodiments, the microbial consortium comprises at least one microorganism from Faecalibacterium prausnitzii species and Eubacterium hallii species.

In some embodiments, the microbial consortium comprises at least one microorganism from Anaerostipes caccae species.

In some embodiments, the microbial consortium comprises at least one microorganism from Lactobacillus johnsonii species, Lactobacillus plantarum species, Lactobacillus reuteri species, Lactobacillus rhamnosus GG species, Lactobacillus acidophilus species, Lactobacillus gallinarum species, Lactobacillus casei species, Lactobacillus paracasei species, Leuconostoc mesenteroides species, Streptococcus thermophiles species and Oenococcus oeni species.

In some embodiments, the microbial consortium comprises at least one microorganism from Bacteroides thetaiotaomicron species, Bacteroides xylanisolvens species, Bacteroides vulgatus species, Bacteroides fragilis species, Roseburia intestinalis species, Ruminococcus champanellensis species and Bifidobacterium adolescentis species.

In some embodiments, the microbial consortium comprises at least one microorganism from Pedobacter heparimis species, Bacteroides eggerthii species, Bacteroides thetaiotaomicron species, Lactobacillus rhamnosus species, Lactobacillus animalis species, Lactobacillus casei species, Enterococcus faecalis species and Alistipes shahii species.

In some embodiments, the microbial consortium comprises at least one microorganism from Bacteroides thetaiotaomicron species, Bacteroides ovatus species and Bacteroides caccae species.

In some embodiments, the microbial consortium comprises at least one microorganism from the Clostridium hiranonis species. Clostridium hiranonis species may be denoted by Taxonomy ID: 89152.

In some embodiments, the microbial consortium comprises at least one microorganism from the Anaerostipes hadrus species. Anaerostipes hadrus species may be denoted by Taxonomy ID: 649756.

In some embodiments, the microbial consortium comprises at least one microorganism from the Bacteroides stercoris species. Bacteroides stercoris species may be denoted by Taxonomy ID: 46506.

In some embodiments, the microbial consortium comprises at least one microorganism from the Phascolarctobacterium succinatutens species. Phascolarctobacterium succinatutens species may be denoted by Taxonomy ID: 626940.

In some embodiments, the microbial consortium comprises at least one microorganism from the Clostridium bolteae species. Clostridium bolteae species may be denoted by Taxonomy ID: 208479.

In some embodiments, the microbial consortium comprises at least one microorganism from the Megamonas hypermegale species. Megamonas hypermegale species may be denoted by Taxonomy ID: 158847.

In some embodiments, the microbial consortium comprises at least one microorganism from the Lactococcus lactis species. Lactococcus lactis species may be denoted by Taxonomy ID: 1358.

In some embodiments, the microbial consortium comprises at least one microorganism from the Clostridium scindens species. Clostridium scindens species may be denoted by Taxonomy ID: 29347.

In some embodiments, the microbial consortium comprises at least one microorganism from the Clostridium hylemonde species. Clostridium hylemonde species may be denoted by Taxonomy ID: 89153.

In some embodiments, the microbial consortium comprises at least one microorganism from the Paraclostridium bifermentans species. Paraclostridium bifermentans species may be denoted by Taxonomy ID: 1490.

In some embodiments, the microbial consortium comprises at least one microorganism from the Clostridium leptum species. Clostridium leptum species may be denoted by Taxonomy ID: 1535.

In some embodiments, the microbial consortium comprises at least one microorganism from the Parabacteroides distasonis species. Parabacteroides distasonis species may be denoted by Taxonomy ID: 823.

In some embodiments, the microbial consortium comprises at least one microorganism from the Eubacterium limosum species. Eubacterium limosum species may be denoted by Taxonomy ID: 1736.

In some embodiments, the microbial consortium comprises at least one microorganism from the Proteocatella sphenisci species. Proteocatella sphenisci species may be denoted by Taxonomy ID: 181070.

In some embodiments, the microbial consortium comprises at least one microorganism from Lachnospiraceae bacterium species. Lachnospiraceae bacterium species may be denoted by Taxonomy ID: 1898203.

In some embodiments, the microbial consortium comprises at least one microorganism from Eubacterium rectale species. Eubacterium rectale species may be denoted by Taxonomy ID: 39491.

In some embodiments, the microbial consortium comprises at least one microorganism from Eubacterium hallii species. Eubacterium hallii species may be denoted by Taxonomy ID: 39488.

In some embodiments, the microbial consortium comprises at least one microorganism from Clostridium sp. SS2/1 species. Clostridium sp. SS2/1 species may be denoted by Taxonomy ID: 411484.

In some embodiments, the microbial consortium comprises at least one microorganism from Clostridium bolteae species. Clostridium bolteae species may be denoted by Taxonomy ID: 208479.

In some embodiments, the microbial consortium comprises at least one microorganism from Clostridium clostridioforme species. Clostridium clostridioforme species may be denoted by Taxonomy ID: 1531.

In some embodiments, the microbial consortium comprises at least one microorganism from Clostridium cellulovorans species. Clostridium cellulovorans species may be denoted by Taxonomy ID: 1493.

In some embodiments, the microbial consortium comprises at least one microorganism from Anaerostipes caccae species. Anaerostipes caccae species may be denoted by Taxonomy ID: 105841.

In some embodiments, the microbial consortium comprises at least one microorganism from Ruminococcus albus species. Ruminococcus albus species may be denoted by Taxonomy ID: 1264.

In the context of the present disclosure, identification of microorganism from a biological sample of a human subject may be done using any conventional method in the microbiology field. For example and without being limited thereto identification of bacteria from a biological sample of a human subject may be done using 16S rRNA (ribosomal RNA) sequencing. Identification of isolated microorganism may be done by conducting similarity analysis between the 16S rRNA gene of the isolated microorganism to different microorganism's 16S rRNA gene sequences available in database. This analysis may be done in order to explore the similarity between a given sequence and all of the available sequences in a database and obtaining the best matched sequences by calculation of a score for the examined similarity. Identity analysis may be conducted by any appropriate program, for example Basic Local Alignment Search Tool (BLAST®) using publicly available databases, for example National Center for Biotechnology Information (NCBI).

It should be noted that the GenBank accession Nos. provide below either provide the 16S rRNA sequence of the microorganism or the sequence of the entire genome. It should be further noted that a skilled person would know to evaluate the 16S rRNA sequence of from the entire genome sequence.

In some embodiments, at least one of the two or more microorganisms comprises 16S rRNA sequences having at least 85%, at least 90%, at least 95%, at least 97%, at least 98%, at least 99% identity with at least one, at least two, at least three, at least four or more nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9. SEQ ID NO:10, SEQ ID NO:11, SEQ ID NO:12, or SEQ ID NO:13.

It should be noted that in the context of the invention, when referring to % identity. each one microorganism from the at least two of the two or more microorganisms can have a different sequence identity from a corresponding sequence denoted above.

In some embodiments, at least one of the two or more microorganisms comprises 16S rRNA sequences having at least 85%, at least 90%, at least 95%, at least 97%, at least 98%, at least 99% identity with at least one nucleic acid sequences denoted by SEQ ID NO:1, SEQ ID NO:9. SEQ ID NO:10 or SEQ ID NO:11.

In some embodiments, at least one of the two or more microorganisms comprises 16S rRNA sequences having at least 85%, at least 90%, at least 95%, at least 97%, at least 98%, at least 99% identity with at least one nucleic acid sequences denoted by SEQ ID NO:2, SEQ ID NO:13 or SEQ ID NO:14.

In some embodiments, at least one of the two or more microorganisms comprises 16S rRNA sequences having at least 85%, at least 90%, at least 95%, at least 97%, at least 98%, at least 99% identity with at least one nucleic acid sequences denoted by SEQ ID NO:1, SEQ ID NO:2, SEQ ID NO:3, SEQ ID NO:4, SEQ ID NO:5 or SEQ ID NO:6.

In some embodiments, at least one of the two or more microorganisms comprises 16S rRNA sequences having, between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5, SEQ ID NO:6, SEQ ID NO:7 SEQ ID NO:8, SEQ ID NO:9. SEQ ID NO:10 or SEQ ID NO:11.

In some embodiments, at least one of the two or more microorganisms comprises 16S rRNA sequences having, between 85% to 99%, at times between 90% to 99%, at times between 95% to 99%, at times between 96% to 99%, at times between 97% to 99%, at times between 98% to 99% identity with at least one nucleic acid sequences denoted by SEQ ID NO:1, SEQ ID NO:2, SEQ ID NO:3, SEQ ID NO:4, SEQ ID NO:5 or SEQ ID NO:6.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:3 and SEQ ID NO:4. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:3 or SEQ ID NO:4.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:5 or SEQ ID NO:6. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:5 or SEQ ID NO:6.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:3 or SEQ ID NO:7. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:3 or SEQ ID NO:7.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2, SEQ ID NO:4 or SEQ ID NO:5. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2, SEQ ID NO:4 or SEQ ID NO:5.

In some embodiments, at least two of the two or more microorganisms comprises 16S rRNA sequences having at least 85%, at least 90%, at least 95%, at least 99% identity with at least two nucleic acid sequences denoted by SEQ ID NO:1, SEQ ID NO:2 or SEQ ID NO:6. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1, SEQ ID NO:2 or SEQ ID NO:6.

In some embodiments, at least two of the two or more microorganisms comprises 16S rRNA sequences having at least 85%, at least 90%, at least 95%, at least 99% identity with at least two nucleic acid sequences denoted by SEQ ID NO:1 and SEQ ID NO:2. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by SEQ ID NO:1 and SEQ ID NO:2.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2 or SEQ ID NO:3. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2 or SEQ ID NO:3.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2 or SEQ ID NO:4. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2 or SEQ ID NO:4.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2 or SEQ ID NO:5. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2 or SEQ ID NO:5.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2, SEQ ID NO:3, SEQ ID NO:4, or SEQ ID NO:5. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2, SEQ ID NO:3, SEQ ID NO:4, or SEQ ID NO:5.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2, SEQ ID NO:4, SEQ ID NO:5, or SEQ ID NO:6. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2, SEQ ID NO:4, SEQ ID NO:5, or SEQ ID NO:6.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2, SEQ ID NO:3, SEQ ID NO:5, or SEQ ID NO:6. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2, SEQ ID NO:3, SEQ ID NO:5, or SEQ ID NO:6.

In some embodiments, at least two of the two or more microorganisms comprises 16S rRNA sequences having at least 85%, at least 90%, at least 95%, at least 99% identity with at least two nucleic acid sequences denoted by one or more of SEQ ID NO:1, SEQ ID NO:2, SEQ ID NO:3, SEQ ID NO:4, or SEQ ID NO:6. In some embodiments, at least two of the two or more microorganisms comprises 16S rRNA sequences having between 85% to 99%, at times between 90% to 99%, at times between 95% to 99%, at times between 96% to 99%, at times between 97% to 99%, at times between 98% to 99% identity with at least two nucleic acid sequences denoted by one or more of SEQ ID NO:1, SEQ ID NO:2, SEQ ID NO:3, SEQ ID NO:4, or SEQ ID NO:6.

The term identity (% identity) as used herein refer to two or more nucleic acid sequences, that are the same. In the context of the present disclosure the sequence identity encompasses transcription changes of DNA to RNA, e.g. T and U are considered identical. The identity may exist over a region of a sequence that is considered by those versed in the art as the variable region of the 16S rRNA. In some embodiments, the identity exists over the length the 16S rRNA or a portion thereof of the variable region.

The % identity between two or more nucleic acid sequences is determined for the two or more sequences when compared and aligned for maximum correspondence. In the context of the present disclosure, sequences (nucleic acid) as described herein having % identity are considered to have the same function/activity of the original sequence to which identity is calculated to.

The threshold sequence identity may be 85%, at times 86%, at times 87%, at times 88%, at times 89%, at times 90%, at times 91%, at times 92%, at times 93%, at times 94%, at times 95%, at times 96%, at times 97%, at times 98%, at times 99% with each one of the % identity denoted herein constitute a separate embodiment of the invention.

In some embodiments, the microbial consortium comprises Clostridium hiranonis DSM 13275. Clostridium hiranonis DSM 13275 may be denoted by Taxonomy ID: 500633. The DNA sequence of Clostridium hiranonis DSM 13275 is provided by GenBank accession No. CP036523. The 16S rRNA sequence of Clostridium hiranonis DSM 13275 is denoted as SEQ ID NO:1.

In some embodiments, the microbial consortium comprises Anaerostipes hadrus DSM 3319. Anaerostipes hadrus DSM 3319 may be denoted by Taxonomy ID: 649757. The 16S rRNA sequence of Anaerostipes hadrus DSM 3319 is provided by GenBank accession No. NR 117139.2 and denoted as SEQ ID NO:2.

In some embodiments, the microbial consortium comprises Bacteroides stercoris ATCC 43183. Bacteroides stercoris ATCC 43183 may be denoted by Taxonomy ID: 449673. The 16S rRNA sequence of Bacteroides stercoris ATCC 43183 is provided by GenBank accession No. NR_112943 and denoted as SEQ ID NO:3.

In some embodiments, the microbial consortium comprises Megamonas hypermegale NCTC10570. Megamonas hypermegale NCTC10570 may be denoted by Taxonomy ID: 1122216. The DNA sequence of Megamonas hypermegale NCTC10570 is provided by GenBank accession No. LT906446.1. The 16S rRNA sequence of Megamonas hypermegale NCTC10570 is denoted as SEQ ID NO:4.

In some embodiments, the microbial consortium comprises Clostridium bolteae ATCC BAA-613. Clostridium bolteae ATCC BAA-613 may be dented by Taxonomy ID: 411902. The DNA sequence of Clostridium bolteae ATCC BAA-613 is provided by GenBank accession No. CP022464.2. The 16S rRNA sequence of Clostridium bolteae ATCC BAA-613 is denoted as SEQ ID NO:5.

In some embodiments, the microbial consortium comprises Lactococcus lactis subsp. cremoris MG1363. Lactococcus lactis subsp. cremoris MG1363 may be denoted by Taxonomy ID: 416870. The DNA sequence of Lactococcus lactis subsp. cremoris MG1363 is provided by GenBank accession No. AM406671.1. The 16S rRNA sequence of Lactococcus lactis subsp. cremoris MG1363 is denoted as SEQ ID NO:6.

In some embodiments, the microbial consortium comprises Phascolarctobacterium succinatutens YIT 12067. Phascolarctobacterium succinatutens YIT 12067 may be denoted by Taxonomy ID: 626939. The 16S rRNA sequence of Phascolarctobacterium succinatutens YIT 12067 is provided by GenBank accession No. AB490811.1 and denoted as SEQ ID NO:7.

In some embodiments, the microbial consortium comprises Bacteroides stercoris CC31F. Bacteroides stercoris CC31F may be denoted by Taxonomy ID: 1073351. The DNA sequence of Bacteroides stercoris CC31F is provided by GenBank accession No. NZ_ATFP00000000.1. The 16S rRNA sequence of Bacteroides stercoris CC31F is denoted as SEQ ID NO:8.

In some embodiments, the microbial consortium comprises Clostridium scindens ATCC 35704. Clostridium scindens ATCC 35704 may be dented by Taxonomy ID: 411468. The 16S rRNA sequence of Clostridium scindens ATCC 35704 is provided by GenBank accession No. NR_028785.1 and is denoted as SEQ ID NO:9.

In some embodiments, the microbial consortium comprises Parabacteroides distasonis CL09T03C24. Parabacteroides distasonis CL09T03C24 may be dented by Taxonomy ID: 999417. The DNA sequence of Parabacteroides distasonis CL09T03C24 is provided by GenBank accession No.: JH976485.1. The 16S rRNA sequence of Parabacteroides distasonis CL09T03C24 is denoted as SEQ ID NO:10.

In some embodiments, the microbial consortium comprises Eubacterium limosum SA11. Eubacterium limosum SA11 may be denoted by Assembly number—GCF 000807675.2. The rRNA sequence of Eubacterium limosum SA11 is provided by GenBank accession No. CP011914.1. The 16S rRNA sequence of Eubacterium limosum strain SA11 is denoted as SEQ ID NO:11.

In some embodiments, the microbial consortium comprises Clostridium sp. SS2/1. Clostridium sp. SS2/1 may be denoted by Taxonomy ID—411484. The rRNA sequence of Clostridium sp. SS2/1 is provided by GenBank accession No. AY305319.1. The 16S rRNA sequence of Clostridium sp. SS2/1 is denoted as SEQ ID NO:12.

In some embodiments, the microbial consortium comprises Lachnospiraceae bacterium 5_1_63FAA. Lachnospiraceae bacterium 5_1_63FAA may be denoted by Taxonomy ID—658089. The rRNA sequence of Lachnospiraceae bacterium 5_1_63FAA is provided by GenBank accession No. GL622435.1. The 16S rRNA sequence of Lachnospiraceae bacterium 5_1_63FAA is denoted as SEQ ID NO:13.

In some embodiments, the microbial consortium comprises two microorganisms, as identified above. In some embodiments, the microbial consortium comprises a combination of two, three, four, five or more microorganisms as identified above.

In some embodiments, at least one of the two or more microorganisms in the microbial consortium comprises 16S rRNA sequences denoted by SEQ ID NO:2.

In some embodiments, at least one of the two or more microorganisms in the microbial consortium comprises 16S rRNA sequences denoted by at least one of SEQ ID NO:2, SEQ ID NO:12, SEQ ID NO:13 or combination thereof.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9. SEQ ID NO:10, SEQ ID NO:11, SEQ ID NO:12 or SEQ ID NO:13.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, SEQ ID NO:5 or SEQ ID NO:6.

In some embodiments, at least one, at times at least two of the two or more microorganisms in the microbial consortium comprises 16S rRNA sequences denoted by SEQ ID NO:1, SEQ ID NO:2, SEQ ID NO:3 or SEQ ID NO:4.

In some embodiments, at least one, at times at least two of the two or more microorganisms in the microbial consortium comprises 16S rRNA sequences denoted by SEQ ID NO:1, SEQ ID NO:2, SEQ ID NO:5 or SEQ ID NO:6.

In some embodiments, at least one, at times at least two of the two or more microorganisms in the microbial consortium comprises 16S rRNA sequences denoted by SEQ ID NO:1, SEQ ID NO:2, SEQ ID NO:3 or SEQ ID NO:7.

In some embodiments, at least one, at times at least two of the two or more microorganisms in the microbial consortium comprises 16S rRNA sequences denoted by SEQ ID NO:1, SEQ ID NO:2, SEQ ID NO:4 or SEQ ID NO:5.

In some embodiments, at least one, at times at least two of the two or more microorganisms in the microbial consortium comprises 16S rRNA sequences denoted by SEQ ID NO:1, SEQ ID NO:2 or SEQ ID NO:6.

In some embodiments, at least one, at times at least two of the two or more microorganisms in the microbial consortium comprises 16S rRNA sequences denoted by SEQ ID NO:1 or SEQ ID NO:2.

In some embodiments, at least one, at times at least two of the two or more microorganisms in the microbial consortium comprises 16S rRNA sequences denoted by SEQ ID NO:1, SEQ ID NO:2 or SEQ ID NO:3.

In some embodiments, at least one, at times at least two of the two or more microorganisms in the microbial consortium comprises 16S rRNA sequences denoted by SEQ ID NO:1, SEQ ID NO:2 or SEQ ID NO:4.

In some embodiments, at least one, at times at least two of the two or more microorganisms in the microbial consortium comprises 16S rRNA sequences denoted by SEQ ID NO:1, SEQ ID NO:2 or SEQ ID NO:5.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, or SEQ ID NO:5.

In some embodiments, at least one, at times at least two of the two or more microorganisms in the microbial consortium comprises 16S rRNA sequences denoted by SEQ ID NO:1, SEQ ID NO:2, SEQ ID NO:4, SEQ ID NO:5, or SEQ ID NO:6.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:5, or SEQ ID NO:6.

In some embodiments, at least one, at times at least two of the two or more microorganisms in the microbial consortium comprises 16S rRNA sequences denoted by SEQ ID NO:1, SEQ ID NO:2, SEQ ID NO:3, SEQ ID NO:4, or SEQ ID NO:6.

In some embodiments, at least one of the two or more microorganisms in the microbial consortium comprises 16S rRNA sequence of SEQ ID NO:2, SEQ ID NO:12 or SEQ ID NO:13 and a 16S rRNA sequence of at least one, at least two, at least three of SEQ ID NO:1, SEQ ID NO:3, SEQ ID NO:4, SEQ ID NO:5, SEQ ID NO:6, SEQ ID NO:7, SEQ ID NO:8, SEQ ID NO:9, SEQ ID NO:10 or SEQ ID NO:11.

In some embodiments, at least one of the two or more microorganisms in the microbial consortium comprises 16S rRNA sequence of SEQ ID NO:2 and a 16S rRNA sequence of at least one, at least two, at least three of SEQ ID NO:1, SEQ ID NO:3, SEQ ID NO:4, SEQ ID NO:5, SEQ ID NO:6, SEQ ID NO:7 or SEQ ID NO:8.

In some embodiments, at least one of the two or more microorganisms in the microbial consortium comprises 16S rRNA sequence of SEQ ID NO:2 or a 16S rRNA sequence of at least one, at least two, at least three of SEQ ID NO:1, SEQ ID NO:3, SEQ ID NO:4, SEQ ID NO:5, SEQ ID NO:6 and SEQ ID NO:7.

In some embodiments, at least one of the two or more microorganisms in the microbial consortium comprises 16S rRNA sequence of SEQ ID NO:2 and a 16S rRNA sequence of at least one, at least two, at least three of SEQ ID NO:1, SEQ ID NO:3, SEQ ID NO:4, SEQ ID NO:5 or SEQ ID NO:6.

In some embodiments, the microbial consortium comprises Anaerostipes hadrus DSM 3319.

In some embodiments, the microbial consortium comprises one or more microorganism selected from Anaerostipes hadrus DSM 3319, Clostridium sp. SS2/1 or Lachnospiraceae bacterium 5_1_63FAA.

In some embodiments, the microbial consortium comprises one or more microorganism selected from Clostridium hiranonis DSM 13275, Clostridium scindens ATCC 35704, Parabacteroides distasonis CL09T03C24 or Eubacterium limosum SA11.

In some embodiments, the microbial consortium comprises one or more microorganism selected from Clostridium hiranonis DSM 13275, Anaerostipes hadrus DSM 3319, Bacteroides stercoris ATCC 43183, Megamonas hypermegale NCTC10570, Clostridium bolteae ATCC BAA-613, Lactococcus lactis subsp. cremoris MG1363, Phascolarctobacterium succinatutens YIT 12067, Bacteroides stercoris CC31F, Clostridium sp. SS2/1 or Lachnospiraceae bacterium 5_1_63FAA, Clostridium scindens ATCC 35704, Parabacteroides distasonis CL09T03C24, Eubacterium limosum SA11 or combination thereof.

In some embodiments, the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, Clostridium bolteae ATCC BAA-613, Lactococcus lactis subsp. cremoris MG1363, Phascolarctobacterium succinatutens YIT 12067, Bacteroides stercoris CC31F or any combination thereof.

In some embodiments, the microbial consortium the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, Clostridium boltede ATCC BAA-613, Lactococcus lactis subsp. cremoris MG1363, Phascolarctobacterium succinatutens YIT 12067 or any combination thereof

In some embodiments, the microbial consortium the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, Clostridium bolteae ATCC BAA-613, Lactococcus lactis subsp. cremoris MG1363 or combination thereof.

In some embodiments, the microbial consortium comprises two or more microorganisms, one of the two or more microorganisms is Anaerostipes hadrus DSM 3319 and one of the two or more microorganisms selected from Clostridium hiranonis DSM 13275, Bacteroides stercoris ATCC 43183, Megamonas hypermegale NCTC10570, Clostridium bolteae ATCC BAA-613, Lactococcus lactis subsp. cremoris MG1363 or a combination thereof

In some embodiments, the microbial consortium the microbial consortium comprises at least two microorganisms selected from Anaerostipes hadrus DSM 3319, Bacteroides stercoris CC31F, Bacteroides stercoris ATCC 43183, Phascolarctobacterium succinatutens YIT 12067, Clostridium boltede ATCC BAA-613, Megamonas hypermegale NCTC10570 Lactococcus lactis subsp. cremoris MG1363 or a combination thereof.

In some embodiments, the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, or combination thereof.

In some embodiments, the microbial consortium comprises at least two microorganisms selected from Clostridium hiranonis DSM 13275, Anaerostipes hadrus DSM 3319, Clostridium bolteae ATCC BAA-613, Lactococcus lactis subsp. cremoris MG1363 or combination thereof.

In some embodiments, the microbial consortium comprises at least two microorganisms selected from Clostridium hiranonis DSM 13275, Anaerostipes hadrus DSM 3319, Bacteroides stercoris ATCC 43183 and Phascolarctobacterium succinatutens YIT 12067 or combination thereof.

In some embodiments, the microbial consortium comprises at least two microorganisms selected from Clostridium hiranonis DSM 13275, Anaerostipes hadrus DSM 3319, Clostridium bolteae ATCC BAA-613, Megamonas hypermegale NCTC10570, or combination thereof.

In some embodiments, the microbial consortium comprises at least two microorganisms selected from Clostridium hiranonis DSM 13275, Anaerostipes hadrus DSM 3319, Lactococcus lactis or combination thereof.

In some embodiments, the microbial consortium comprises at least two microorganisms selected from Clostridium hiranonis DSM 13275 and Anaerostipes hadrus DSM 3319.

In some embodiments, the microbial consortium comprises at least two microorganisms selected from Clostridium hiranonis DSM 13275, Anaerostipes hadrus DSM 3319 and Bacteroides stercoris ATCC 43183, subspecies or combination thereof.

In some embodiments, the microbial consortium comprises at least two microorganisms selected from Clostridium hiranonis DSM 13275, Anaerostipes hadrus DSM 3319, Megamonas hypermegale NCTC10570, or combination thereof.

In some embodiments, the microbial consortium comprises at least two microorganisms selected from Clostridium hiranonis DSM 13275, Anaerostipes hadrus DSM 3319, Clostridium boltede ATCC BAA-613 or combination thereof.

In some embodiments, the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, Clostridium bolteae ATCC BAA-613 or combination thereof.

In some embodiments, the microbial consortium comprises at least two microorganisms selected from Clostridium hiranonis DSM 13275, Anaerostipes hadrus DSM 3319, Megamonas hypermegale NCTC10570, Clostridium boltede ATCC BAA-613, Lactococcus lactis subsp. cremoris MG1363 or combination thereof.

In some embodiments, the microbial consortium comprises at least two microorganisms selected from Clostridium hiranonis DSM 13275, Anaerostipes hadrus DSM 3319, Bacteroides stercoris ATCC 43183, Clostridium boltede ATCC BAA-613, Lactococcus lactis subsp. cremoris MG1363 or combination thereof.

In some embodiments, the microbial consortium comprises at least two microorganisms selected from Clostridium hiranonis DSM 13275, Anaerostipes hadrus DSM 3319, Bacteroides stercoris ATCC 43183, Megamonas hypermegale NCTC10570, Lactococcus lactis subsp. cremoris MG1363 or combination thereof.

In some embodiments, the microbial consortium comprises two microorganisms, as identified above. In some embodiments, the microbial consortium comprises a combination of two or three microorganisms, as identified above. In some embodiments, the microbial consortium comprises a combination of two, three or four microorganisms, as identified above. In some embodiments, the microbial consortium comprises a combination of two, three, four, five, six or more microorganisms as identified above.

In some embodiments, the microbial consortium comprises at least two isolated or purified microorganisms belonging to the genus, spices or strain identified by NCBI Taxonomy IDs selected from the group consisting of NCBI Taxonomy ID: 1485, 207244, 816, 33024, 158846, 1357, 375288, 1730, 186803, 181069, 1263, 216851, 841, 580596, 1578, 1243, 1301, 46254, 1678, 89152, 649756, 46506, 626940, 208479, 158847, 1358, 29347, 89153, 1490, 1535, 823, 1736, 181070, 1898203, 39491, 39488, 411484, 208479, 1531, 1493, 105841, 1264, 500633, 649757, 449673, 1122216, 411902, 416870, 626939, 1073351, 411468, 999417 and 658089.

In some aspects, which may be implemented as certain embodiments of the microbial consortium of the invention, it is provided as microbial consortium comprising two or more purified or isolated microorganisms, at least one of the two or more microorganisms is capable of modulating the production of PE, AEA or a combination thereof and at least one different microorganism of the two or more microorganisms is a microorganism belonging to species selected from Clostridium hiranonis species, Anaerostipes hadrus species, Bacteroides stercoris species, Megamonas hypermegale species, Clostridium bolteae species and Lactococcus lactis subspecies or combination thereof.

In some aspects, which may be implemented as certain embodiments of the microbial consortium of the invention, it is provided as microbial consortium comprising two or more purified or isolated microorganisms, at least one of the two or more microorganisms is capable of modulating the production of PE, AEA or a combination thereof and at least one different microorganism of the two or more microorganisms comprises a 16S rRNA sequences having at least 85%, at least 90%, at least 95%, at least 97%, at least 98%, at least 99% identity with at least one nucleic acid sequences denoted by SEQ ID NO:1, SEQ ID NO:2, SEQ ID NO:3, SEQ ID NO:4, SEQ ID NO:5, SEQ ID NO:6 or a combination thereof.

In some aspects, which may be implemented as certain embodiments of the microbial consortium of the invention, it is provided as microbial consortium comprising two or more purified or isolated microorganisms, at least one of the two or more microorganisms is capable of modulating the production of PE, AEA or a combination thereof and at least one different microorganism of the two or more microorganisms comprises a 16S rRNA sequences denoted by SEQ ID NO:1, SEQ ID NO:2, SEQ ID NO:3, SEQ ID NO:4, SEQ ID NO:5, SEQ ID NO:6 or a combination thereof.

In some aspects, which may be implemented as certain embodiments of the microbial consortium of the invention, it is provided as microbial consortium comprising two or more purified or isolated microorganisms, at least one of the two or more microorganisms is capable of modulating the production of PE, AEA or a combination thereof and at least one different microorganism of the two or more microorganisms is selected from Clostridium hiranonis DSM 13275, Anaerostipes hadrus DSM 3319, Bacteroides stercoris ATCC 43183, Megamonas hypermegale NCTC10570, Clostridium boltede ATCC BAA-613, Lactococcus lactis subsp. cremoris MG1363, or any combination thereof.

In some aspects, which may be implemented as certain embodiments of the microbial consortium of the invention, it is provided as microbial consortium comprising two or more purified or isolated microorganisms, at least one of the two or more microorganisms is selected from Anaerostipes hadrus species and at least one different microorganism of the two or more microorganisms is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

In some aspects, which may be implemented as certain embodiments of the microbial consortium of the invention, it is provided as microbial consortium comprising two or more purified or isolated microorganisms, at least one of the two or more microorganisms comprises 16S rRNA sequences having at least 85%, at least 90%, at least 95%, at least 97%, at least 98%, at least 99% identity with at least one nucleic acid sequences denoted by SEQ ID NO:2 and at least one different microorganism of the two or more microorganisms is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

In some aspects, which may be implemented as certain embodiments of the microbial consortium of the invention, it is provided as microbial consortium comprising two or more purified or isolated microorganisms, at least one of the two or more microorganisms comprises 16S rRNA sequences denoted by SEQ ID NO:2 and at least one different microorganism of the two or more microorganisms is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

In some aspects, which may be implemented as certain embodiments of the microbial consortium of the invention, it is provided as microbial consortium comprising two or more purified or isolated microorganisms, at least one of the two or more microorganisms is Anaerostipes hadrus DSM 3319 and at least one different microorganism of the two or more microorganisms is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

In some embodiments, the microbial consortium comprises Clostridium hiranonis DSM 13275, Anaerostipes hadrus DSM 3319, Bacteroides stercoris ATCC 43183 and Megamonas hypermegale NCTC10570.

In some embodiments, the microbial consortium consists of Clostridium hiranonis DSM 13275, Anaerostipes hadrus DSM 3319, Bacteroides stercoris ATCC 43183 and Megamonas hypermegale NCTC10570. This microbial consortium including the four listed strains, as purified or isolated bacteria, is referred herein at times as Consortium 1 (“Cons. #1 or BMC321).

In some embodiments, the microbial consortium comprises Clostridium hiranonis DSM 13275, Anaerostipes hadrus DSM 3319, Clostridium bolteae ATCC BAA-613 and Lactococcus lactis subsp. cremoris MG1363.

In some embodiments, the microbial consortium consists of Clostridium hiranonis DSM 13275, Anaerostipes hadrus DSM 3319, Clostridium bolteae ATCC BAA-613 and Lactococcus lactis subsp. cremoris MG1363. This microbial consortium including the four listed strains, as purified or isolated bacteria, is referred herein at times as Consortium 2 (“Cons. #2 or BMC322).

In some embodiments, the microbial consortium comprises Clostridium hiranonis DSM 13275, Anaerostipes hadrus DSM 3319, Clostridium boltede ATCC BAA-613, Bacteroides stercoris ATCC 43183 and Phascolarctobacterium succinatutens YIT 12067.

In some embodiments, the microbial consortium consists of Clostridium hiranonis DSM 13275, Anaerostipes hadrus DSM 3319, Clostridium bolteae ATCC BAA-613, Bacteroides stercoris ATCC 43183 and Phascolarctobacterium succinatutens YIT 12067. This microbial consortium including the four listed strains, as purified or isolated bacteria, is referred herein at times as Consortium 3 (“Cons. #3).

In some embodiments, the microbial consortium comprises Clostridium hiranonis DSM 13275, Anaerostipes hadrus DSM 3319, Clostridium boltede ATCC BAA-613, and Megamonas hypermegale NCTC10570.

In some embodiments, the microbial consortium consists of Clostridium hiranonis DSM 13275, Anaerostipes hadrus DSM 3319, Clostridium bolteae ATCC BAA-613, and Megamonas hypermegale NCTC10570. This microbial consortium including the three listed strains, as purified or isolated bacteria, is referred herein at times as Consortium 4 (“Cons. #4).

In some embodiments, the microbial consortium comprises Clostridium hiranonis DSM 13275, Anaerostipes hadrus DSM 3319, and Lactococcus lactis subsp. cremoris MG1363.

In some embodiments, the microbial consortium consists of Clostridium hiranonis DSM 13275, Anaerostipes hadrus DSM 3319, and Lactococcus lactis subsp. cremoris MG1363. This microbial consortium including the three listed strains, as purified or isolated bacteria, is referred herein at times as Consortium 5 (“Cons. #5).

In some embodiments, the microbial consortium comprises Clostridium hiranonis DSM 13275 and Anderostipes hadrus DSM 3319.

In some embodiments, the microbial consortium consists of Clostridium hiranonis DSM 13275 and Anaerostipes hadrus DSM 3319. This microbial consortium including the two listed strains, as purified or isolated bacteria, is referred herein at times as Consortium 6 (“Cons. #6).

In some embodiments, the microbial consortium comprises Clostridium hiranonis DSM 13275, Anaerostipes hadrus DSM 3319 and Bacteroides stercoris ATCC 43183.

In some embodiments, the microbial consortium consists of Clostridium hiranonis DSM 13275, Anaerostipes hadrus DSM 3319 and Bacteroides stercoris ATCC 43183. This microbial consortium including the three listed strains, as purified or isolated bacteria, is referred herein at times as Consortium 7 (“Cons. #7).

In some embodiments, the microbial consortium comprises Clostridium hiranonis DSM 13275, Anaerostipes hadrus DSM 3319 and Megamonas hypermegale NCTC10570.

In some embodiments, the microbial consortium consists of Clostridium hiranonis DSM 13275, Anaerostipes hadrus DSM 3319 and Megamonas hypermegale NCTC10570. This microbial consortium including the three listed strains, as purified or isolated bacteria, is referred herein at times as Consortium 8 (“Cons. #8).

In some embodiments, the microbial consortium comprises Clostridium hiranonis DSM 13275, Anaerostipes hadrus DSM 3319 and Clostridium bolteae ATCC BAA-613.

In some embodiments, the microbial consortium consists of Clostridium hiranonis DSM 13275, Anaerostipes hadrus DSM 3319 and Clostridium boltede ATCC BAA-613. This microbial consortium including the three listed strains, as purified or isolated bacteria, is referred herein at times as Consortium 9 (“Cons. #9).

In some embodiments, the microbial consortium comprises Clostridium hiranonis DSM 13275, Anaerostipes hadrus DSM 3319 Bacteroides stercoris ATCC 43183, Megamonas hypermegale NCTC10570, and Clostridium bolteae ATCC BAA-613.

In some embodiments, the microbial consortium consists of Clostridium hiranonis DSM 13275, Anaerostipes hadrus DSM 3319 Bacteroides stercoris ATCC 43183, Megamonas hypermegale NCTC10570, and Clostridium bolteae ATCC BAA-613. This microbial consortium including the five listed strains, as purified or isolated bacteria, is referred herein at times as Consortium 10 (“Cons. #10).

In some embodiments, the microbial consortium comprises Clostridium hiranonis DSM 13275, Anaerostipes hadrus DSM 3319 Megamonas hypermegale NCTC10570, Clostridium boltede ATCC BAA-613 and Lactococcus lactis subsp. cremoris MG1363.

In some embodiments, the microbial consortium consists of Clostridium hiranonis DSM 13275, Anaerostipes hadrus DSM 3319 Megamonas hypermegale NCTC10570, Clostridium boltede ATCC BAA-613 and Lactococcus lactis subsp. cremoris MG1363. This microbial consortium including the five listed strains, as purified or isolated bacteria, is referred herein at times as Consortium 11 (“Cons. #11).

In some embodiments, the microbial consortium comprises Clostridium hiranonis DSM 13275, Anaerostipes hadrus DSM 3319, Bacteroides stercoris ATCC 43183, Clostridium bolteae ATCC BAA-613 and Lactococcus lactis subsp. cremoris MG1363.

In some embodiments, the microbial consortium consists of Clostridium hiranonis DSM 13275, Anaerostipes hadrus DSM 3319, Bacteroides stercoris ATCC 43183, Clostridium bolteae ATCC BAA-613 and Lactococcus lactis subsp. cremoris MG1363. This microbial consortium including the five listed strains, as purified or isolated bacteria, is referred herein at times as Consortium 12 (“Cons. #12).

In some embodiments, the microbial consortium comprises Clostridium hiranonis DSM 13275, Anaerostipes hadrus DSM 3319, Bacteroides stercoris ATCC 43183, Megamonas hypermegale NCTC10570 and Lactococcus lactis subsp. cremoris MG1363.

In some embodiments, the microbial consortium consists of Clostridium hiranonis DSM 13275, Anaerostipes hadrus DSM 3319, Bacteroides stercoris ATCC 43183, Megamonas hypermegale NCTC10570 and Lactococcus lactis subsp. cremoris MG1363. This microbial consortium including the five listed strains, as purified or isolated bacteria, is referred herein at times as Consortium 13 (“Cons. #13).

In another aspect of the invention, which may be implemented as certain embodiments of the microbial consortium of the invention, provided is a microbial consortium selected from the group consisting of Consortium 1, Consortium 2, Consortium 3, Consortium 4, Consortium 5, Consortium 6, Consortium 7, Consortium 8, Consortium 9, Consortium 10, Consortium 11, Consortium 12 and Consortium 13.

In another aspect of the invention, which may be implemented as certain embodiments of the microbial consortium of the invention, provided is a microbial consortium selected from Consortium 1 and Consortium 2.

In some embodiments, the microbial consortium comprises the same or equivalent amounts of the microorganisms forming the consortium.

In some further embodiments, the microbial consortium comprises different amounts of the microorganisms forming the consortium.

In some embodiments, the total number per one dose (cell count/amount/colony forming units-CFUs/Optical density measurement) of each microorganism forming the microbial consortium is at least 1×10². at least 1×10³, at least 10⁴, at least 1×10⁵, at times at least 1×10⁶, at times at least 1×10⁷, at times at least 1×10⁸.

In some embodiments, the total number per one dose (cell count/amount/colony forming units-CFUs/Optical density measurement) of each microorganism forming the microbial consortium between about 1×10⁸ and about 5×10¹⁰ CFU, between about 2×10⁸ and about 4×10¹⁰, between about 3×10⁸ and about 3×101⁰, between about 5×10⁸ and about 1×10¹⁰.

In some embodiments, the microbial consortium comprises the same or equivalent amounts of the microorganisms forming the consortium.

In some further embodiments, the microbial consortium comprises different amounts of the microorganisms forming the consortium. In some embodiments, the total number (cell count/amount/colony forming units-CFUs) of each one of microorganisms forming the microbial consortium is between about 1×10³ to about 1×10¹², between 1×10⁵ to about 1×10¹⁰, 1×10⁸ to about 5×10¹⁰, between 2×10⁸ to about 4×10¹⁰, between 3×10⁸ to about 3×10¹⁰, between 5×10⁸ to about 1×10¹⁰.

In some embodiments, the total number (cell count/amount/colony forming units-CFUs) of microorganisms forming the microbial consortium is between about 1×10³ to about 1×10¹², between 1×10⁵ to about 1×10¹⁰, 1×10⁸ to about 5×10¹⁰, between 2×10⁸to about 4×10¹⁰, between 3×10⁸ to about 3×10¹⁰, between 5×10⁸ to about 1×10¹⁰.

As detailed above, the two or more microorganisms can be identified in, purified or isolated from the microbiome of a reference subject, for example by collecting a biological sample. The biological sample may be any sample from which the population of microorganisms can be isolated, for example, faeces. In yet another embodiment, the sample may be a biopsy of human organs or tissue, specifically, a gut biopsy.

The microbial consortium may be formulated in a variety of forms, depending on the storage, administration etc. Non-limiting forms include solid, dry form, for example, in a lyophilized powder, gel form, a suspension, a cell lysate or extract. In some embodiments, the microbial consortium may be suspended in a liquid medium (such as PBS or saline) and used in a suspension form.

The microbial consortium of the invention may be used in the preparation of a pharmaceutical formulation/compositions/suspension or in the manufacture of a formulation/compositions/suspension for use in treatment.

As such, formulation/compositions/suspension of the invention may comprise apart from a therapeutically effective amount of a microbial consortium of the invention, at least one additional component as detailed herein.

In a further aspect, the invention relates to a composition (suspension or formulation) comprising microbial consortium of the invention.

In accordance with some embodiments, the compositions of the invention comprising a microbial consortium comprising two or more microorganisms, the two or more microorganisms are capable of modulating a phospholipid and/or AEA and modulating at least one of (i) short chain fatty acid (SCFA), (ii) secondary bile acid, (iii) a polysaccharide, (iv) a GAG and (v) lactate.

As described herein, the microbial consortium and/or the suspension/composition comprising the same may form a kit of the invention. In general, the composition and/or the suspension and/or kit described herein comprising the microbial consortium as well as the microbial consortium per se form part of this invention. It should be noted that the forms described herein for the microbial consortium per se apply for the composition and/or the suspension and/or kit comprising the microbial consortium.

In yet some further embodiments, the composition of the invention may optionally further comprise at least one of pharmaceutically acceptable carrier/s, excipient/s, additive/s diluent/s and adjuvant/s. As used herein “pharmaceutically acceptable carrier” includes any and all solvents, dispersion media, coatings and the like.

Aqueous suspensions may further contain substances which increase the viscosity of the suspension including, for example, sodium carboxymethylcellulose, sorbitol and/or dextran. The suspension may also contain stabilizers.

It should be understood that in addition to the ingredients particularly mentioned above, the formulations may also include other agents conventional in the art having regard to the type of formulation in question.

The microbial consortium of the invention and/or any suspensions/compositions comprising the same can be administered and dosed by the methods of the invention as described below, in accordance with medical procedures known in the art. For example, the suspensions/compositions used in the methods and kits of the invention, described herein below, may be adapted for administration by various modes of interferon administration are known in the art including, for example, systemic, parenteral, intraperitoneal, transdermal, oral (including buccal or sublingual), rectal, topical (including buccal or sublingual), vaginal, intranasal and any other appropriate routes. Specific examples include but not limited to, injection (e.g., using a subcutaneous, intramuscular, intravenous, or intradermal injection), intranasal administration and oral administration.

In some embodiments, the microbial consortium of the invention and/or any suspensions/compositions comprising the same may be formulated for oral administration.

In some embodiments, the microbial consortium of the invention and/or any suspensions/compositions comprising the same may be formulated for delivery to the intestine. In some embodiments, the microbial consortium of the invention and/or any suspensions/compositions comprising the same may be formulated into food or a beverage. In some embodiments, the microbial consortium of the invention and/or any suspensions/compositions comprising the same may be formulated to be contained within a carrier.

In some embodiments, the microbial consortium of the invention and/or any suspensions/compositions comprising the same is enterically coated.

The microbial consortium, the compositions comprising the microbial consortium of the present invention and the kits of the invention may be useful for a variety of purposes, including for treatment of subjects in need of a treatment by the microbial consortium of the invention. Specifically, the microbial consortium of the invention may be for use to treat a disease treatable by the microbial consortium.

In some embodiments, the types of the at least two microorganisms and the amount of each one of the at least two microorganisms in the microbial consortium, may be adapted based on the disorder to be treated or the severity of the disorder. For example, prior to treatment, diagnostics of the disorder and the severity may determine these parameters.

In accordance with some other embodiments, the microbial consortium described herein may be useful in affecting the immune system or the gastrointestinal system of a host subject and hence treat a subject being diagnosed with an immune-related condition.

Thus, the present invention further provides a microbial consortium as detailed above for use in the treatment of a subject suffering from an immune-related condition, specifically an inflammatory condition and more specifically an inflammatory disease of the gut.

In some other embodiments, the microbial consortium is for use in treating a subject being diagnosed with a disease that may be treatable by the consortium, such as an immune-related condition.

Thus, the present invention further provides a microbial consortium according to the invention comprising two or more purified or isolated microorganisms of the invention, compositions of the invention or kits of the invention for use in a method of treating of a subject suffering from an immune-related condition, specifically an inflammatory condition.

In some embodiments the microbial consortium, composition or kits comprising the same may be administrated in combination with an anti-inflammatory treatment. The microbial consortium and the anti-inflammatory treatment may be administrated simultaneously or sequentially.

In some embodiments the anti-inflammatory treatment is an anti-inflammatory drug.

The anti-inflammatory drug is at least one of corticosteroid or aminosalicylate.

In some embodiments, the corticosteroid is at least one of hydrocortisone, methylprednisolone, prednisone, prednisolone or budesonide.

In some embodiments, the aminosalicylate is at least one of mesalamine, sulfasalazine, balsalazide or olsalazine.

In some embodiments, the anti-inflammatory treatment may be an immune system suppressor.

In some embodiments, the immune system suppressor is at least one of azathioprine, mercaptopurine, cyclosporine, methotrexate or tacrolimus.

In some embodiments, the anti-inflammatory treatment is an immune system modulator.

In some embodiments, the immune system modulator is a biological treatment. In some embodiments, the immune system modulator is at least one of infliximab (Remicade), adalimumab (Humira), golimumab (Simponi), natalizumab (Tysabri), vedolizumab (Entyvio) or ustekinumab (Stelara).

In some embodiments, the anti-inflammatory treatment is an antibiotic drug. An example of an antibiotic drug includes ciprofloxacin or metronidazole.

As shown in the examples below the microbial consortium of the invention were effective in preventing and treating an inflammatory condition of the gut.

Specifically, as shown in Example 2 providing results from Dextran Sodium Sulfate (DSS)-induced colitis in mice model, microbial consortium of the invention administered to mice a week prior to DSS administration were effective in preventing inflammation as well as in increasing recovery. In addition, and as shown in Example 3, microbial consortium of the invention has a therapeutic effect on the induced inflammatory disease.

Hence, it was suggested that the microbial consortium of the invention can be used in method of preventing and treating an inflammatory condition, specifically an inflammatory condition of the gut (colitis).

Thus, in yet another aspect, the invention provides a method for treating a disorder in a subject in need thereof. In some embodiments, the method for treating, preventing, ameliorating, reducing or delaying the onset of an immune-related condition in a subject in need thereof comprising administering to such subject a therapeutically effective amount of a microbial consortium of the invention or any composition or kit comprising the same.

As used herein, an immune-related condition is a condition that is associated with the immune system of a subject, either through activation or inhibition of the immune system, or that can be treated, prevented or diagnosed by targeting a certain component of the immune response in a subject, such as the adaptive or innate immune response.

In some embodiments, the immune-related condition may be any one of an autoimmune condition or an inflammatory condition.

In some embodiments, the immune-related condition is an autoimmune condition.

Non-limiting examples for autoimmune condition include Multiple Sclerosis (MS), inflammatory arthritis. rheumatoid arthritis (RA), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, Behget's syndrome, ankylosing spondylitis, pemphigus, bullous pemphigoid, dermatitis herpetiformis, inflammatory bowel disease (IBD), ulcerative colitis and Crohn's disease and fatty liver disease.

In some embodiments, the immune-related condition is an inflammatory condition.

As used herein, an inflammatory condition refers to any disease or pathologically condition which can benefit from the reduction of at least one inflammatory parameter, for example, induction of an inflammatory cytokine such as IFN-gamma and IL-2. The condition may be caused (primarily) from inflammation, or inflammation may be one of the manifestations of the diseases caused by another physiological cause.

In some other embodiments, the inflammatory condition is an acute inflammatory condition.

In some embodiments, the inflammatory condition is a chronic inflammatory condition.

The inflammatory condition is not an infectious condition caused by a pathogenic agent. In other words, the inflammatory condition is a non-infectious inflammatory condition.

As used herein, the term “non-infectious inflammatory disorders” used herein is to be understood as encompassing any immune response that is not related to activation of the immune system, e.g. by an infection. Such non-infectious inflammatory disorders denote any disorder which the activation of macrophages or activated macrophages play a role such as auto-immune disorders and inflammatory disorders which are not infection related, i.e. non-pathogenic, caused by other than an infectious agent (e.g. auto-antigen, hypersensitivity, wound).

Non-limiting examples of inflammatory disorders are inflammatory diseases of the gastrointestinal tract, inflammatory diseases of the skin, inflammatory diseases of the respiratory system, inflammatory diseases of the musculoskeletal system, inflammatory diseases of the kidneys, inflammatory diseases of the nervous system or inflammatory conditions of the cardiovascular system, such as myocardial infarction, myocarditis, atherosclerosis, hypertensive cardiomyopathy, atheroma, intimal hyperplasia or restenosis

In some embodiments, the inflammatory condition is an inflammatory condition of the gastrointestinal tract (gut).

In some embodiments, the inflammatory condition of the gastrointestinal tract is one or more of Crohn's disease, inflammatory bowel disease, gastritis, colitis, ulcerative colitis, irritable bowel syndrome, gastric ulcer, duodenal ulcer or combination thereof.

In some embodiments, the inflammatory condition of the skin is psoriasis.

In some embodiments, the inflammatory condition of the respiratory system is one or more of asthma, allergic rhinitis or chronic obstructive pulmonary disease (COPD), pulmonary fibrosis, sarcoidosis, or combination thereof.

In some embodiments, the inflammatory diseases of the musculoskeletal system is one or more of rheumatoid arthritis, osteomyelitis, osteoporosis, or neuritis, systemic sclerosis, or combination thereof.

In some embodiments, the inflammatory diseases of the kidneys is one or more of glomerulonephritis, renal ischemia, or renal inflammation.

In some embodiments, the inflammatory diseases of the nervous system is one or more of multiple sclerosis, Alzheimer's disease and HIV-1-associated dementia, or combination thereof.

In some embodiments, the inflammatory conditions of the cardiovascular system, is one or more of myocardial infarction, myocarditis, atherosclerosis, hypertensive cardiomyopathy, atheroma, intimal hyperplasia, restenosis or combination thereof.

In some embodiments, an inflammatory condition that may be applicable for the present application may be an inflammatory disease of the gastrointestinal tract. In some embodiments, the inflammatory condition of the gastrointestinal tract is IBD.

In some embodiments, the inflammatory condition of the gastrointestinal tract is ulcerative colitis, Crohn's disease or combination thereof.

In some embodiments, the inflammatory condition of the gastrointestinal tract is ulcerative colitis.

In some embodiments, the inflammatory condition of the gastrointestinal tract is Crohn's disease.

In accordance with the methods of the invention, administering the effective amount of a microbial consortium of the invention ameliorates one or more signs or symptoms of the inflammatory condition. In other words, the methods of the invention are for treating disorders may be treatable with the microbial consortium of the invention.

In an another aspect, the invention provides a method of treating, preventing, ameliorating, reducing or delaying the onset of an inflammatory condition, specifically a inflammatory condition of the gut, in a human subject in need thereof comprising the step of administering to the subject an effective amount of a microbial consortium comprising two or more isolated microorganism or purified microorganisms, at least two microorganisms in the microbial consortium is characterized by having one or more of (i) producing a phospholipid, specifically PE, (ii) modulating the production of endocannabinoid, specifically AEA, (iii) producing lactate, (iv) producing acetic acid, (v) producing butyric acid, (vi) producing propionic acid, (vii) producing at least one bile acid, specifically DOC, LCA or any combination thereof, (viii) degrading at least one polysaccharide, (ix) degrading at least one GAG or (x) any combination thereof. In such embodiments, the method is for treating, preventing, ameliorating, reducing or delaying the onset of an inflammatory condition of the gut, specifically IBD, more specifically, ulcerative colitis, Crohn's disease or combination thereof.

In some embodiments, the methods of the invention comprising the step of administering to the subject an effective amount of a microbial consortium comprising two or more isolated microorganism or purified microorganisms at least one microorganism in the microbial consortium is characterized by having one or more of (i) producing a phospholipid, specifically PE, (ii) modulating the production of an endocannabinoid in the host, specifically AEA or (iii) any combination thereof and at least one other microorganism in the microbial consortium is characterized by having one or more of (i) producing lactate, (ii) producing acetic acid, (ii) producing butyric acid, (iv) producing propionic acid, (v) producing at least one bile acid, specifically DOC, LCA or any combination thereof, (vi) degrading at least one polysaccharide, (vii) degrading at least one GAG or (viii) any combination thereof.

In some embodiments, the methods of the invention comprising the step of administering to the subject an effective amount of a microbial consortium comprising two or more isolated microorganism or purified microorganisms capable of having one or more of (i) activating Tregs, (ii) activating an anti-inflammatory cytokine, (iii) activating a bile acid receptor (iv) activating gut barrier integrity, (v) inhibiting NF-κB, (vi) inhibiting an inflammasome, (vii) inhibiting a pro-inflammatory cytokine or (viii) any combination thereof.

In some embodiments, the methods of the invention comprises administration of a therapeutically effective amount of a microbial consortium comprises at least two microorganisms species belonging to species selected from Clostridium hiranonis species, Anaerostipes hadrus species, Bacteroides stercoris species, Megamonas hypermegale species, Clostridium bolteae species, Lactococcus lactis species, subspecies or combination thereof.

In some embodiments, the methods of the invention comprises administration of a therapeutically effective amount of a microbial consortium comprises two or more microorganism having 16S rRNA sequences with at least 85%, at least 90%, at least 95%, at least 96%, at least 97%, at least 98%, at least 99%, at least 99.5% identity with one or more nucleic acid sequences denoted by SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9. SEQ ID NO:10, SEQ ID NO:11, SEQ ID NO:12, SEQ ID NO:13 or a combination thereof.

In some embodiments, the methods of the invention comprises administration of a therapeutically effective amount of a microbial consortium comprises two or more microorganism having 16S rRNA sequences with at least 85%, at least 90%, at least 95%, at least 96%, at least 97%, at least 98%, at least 99%, at least 99.5% identity with at least one nucleic acid sequences denoted by SEQ ID NO:1, SEQ ID NO:2, SEQ ID NO:3, SEQ ID NO:4, SEQ ID NO:5, SEQ ID NO:6 or a combination thereof.

In some embodiments, the methods of the invention comprises administration to the subject a therapeutically effective amount of a microbial consortium comprising two or more purified or isolated microorganisms having a 16S rDNA sequence that is at least 95%, at least 96%, at least 97%, at least 98%, at least 99%, at least 99.5% identical to one, two, three, four, five, six seven, eight 16S rDNA sequence listed in Table 1.

In some embodiments, the methods of the invention comprises administration of a therapeutically effective amount of a microbial consortium comprises one, two, three, four or five microorganisms selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9. SEQ ID NO:10, SEQ ID NO:11, SEQ ID NO:12, SEQ ID NO:13, or a combination thereof.

some embodiments, the methods of the invention comprises administration of a therapeutically effective amount of a microbial consortium comprises one or more microorganisms selected from SEQ ID NO:1, SEQ ID NO:2, SEQ ID NO:3, SEQ ID NO:4, SEQ ID NO:5, SEQ ID NO:6 or a combination thereof.

In some embodiments, the methods of the invention comprises administration of a therapeutically effective amount of a microbial consortium comprises one or more microorganisms selected from Clostridium hiranonis DSM 13275, Anaerostipes hadrus DSM 3319, Bacteroides stercoris ATCC43183, Megamonas hypermegale NCTC10570, Clostridium bolteae ATCC BAA-613, Lactococcus lactis subsp. cremoris MG1363 or a combination thereof.

In some embodiments, the methods of the invention comprises administration to the subject a therapeutically effective amount of a microbial consortium being at least one of Consortium 1, Consortium 2, Consortium 3, Consortium 4, Consortium 5, Consortium 6, Consortium 7, Consortium 8, Consortium 9. Consortium 10, Consortium 11, Consortium 12 and Consortium 13.

In some embodiments, the methods of the invention comprise administration to the subject a therapeutically effective amount of a microbial consortium being at least one of Consortium 1 or Consortium 2.

In such embodiments of the methods of the invention, the microbial consortium of the invention is administered for treating, preventing, ameliorating, reducing or delaying the onset of an inflammatory condition, specifically a inflammatory condition of the gut, specifically IBD, more specifically, ulcerative colitis, Crohn's disease or combination thereof.

In another aspect of the invention, which may be implemented as certain embodiments of the methods of the invention, provided is a method for treating, preventing, ameliorating, reducing or delaying the onset of an inflammatory condition, in a human subject in need thereof comprising administration a therapeutically effective amount of the microbial consortium of the invention with a therapeutically effective amount of an anti-inflammatory treatment or immune modulation/suppression treatment.

In accordance with some embodiments the method comprising administrating to the subject a therapeutically effective amount of the microbial consortium of the invention and a therapeutically effective amount of the anti-inflammatory treatment simultaneously or sequentially.

In accordance with some embodiments the method comprising administrating to the subject a therapeutically effective amount of the microbial consortium of the invention and a therapeutically effective amount of the anti-inflammatory treatment concurrently. In some embodiments the anti-inflammatory treatment is an anti-inflammatory drug.

The anti-inflammatory drug is at least one of corticosteroid or aminosalicylate.

In some embodiments, the corticosteroid is at least one of hydrocortisone, methylprednisolone, prednisone, prednisolone or budesonide.

In some embodiments, the aminosalicylate is at least one of mesalamine, sulfasalazine, balsalazide or olsalazine.

In some embodiments, the anti-inflammatory treatment may be an immune system suppressor.

In some embodiments, the immune system suppressor is at least one of azathioprine, mercaptopurine, cyclosporine, methotrexate or tacrolimus.

In some embodiments, the anti-inflammatory treatment is an immune system modulator.

In some embodiments, the immune system modulator is a biological treatment. In some embodiments, the immune system modulator is at least one of infliximab (Remicade), adalimumab (Humira), golimumab (Simponi), natalizumab (Tysabri), vedolizumab (Entyvio) or ustekinumab (Stelara).

In some embodiments, the anti-inflammatory treatment is an antibiotic drug. An example of an antibiotic drug includes ciprofloxacin or metronidazole.

In some embodiments of the methods of the invention for treating, preventing, ameliorating, reducing or delaying the onset of IBD, ulcerative colitis, Crohn's disease or combination thereof, comprising administrating to the subject in need thereof a therapeutically effective amount of the microbial consortium of the invention.

In some embodiments of the methods of the invention for treating, preventing, ameliorating, reducing or delaying the onset of IBD, ulcerative colitis, Crohn's disease or combination thereof, comprising administrating to the subject in need thereof a therapeutically effective amount of the microbial consortium of the invention and a therapeutically effective amount of the anti-inflammatory treatment, the method comprising administrating a therapeutically effective amount of the microbial consortium with a therapeutically effective amount of an anti-inflammatory treatment as detailed herein.

The methods of the invention are applicable to an individual at any age. In some embodiments, the methods of the invention are applicable for children at age up to 18 years, at time up to 16 years, at times up to 13 years. In some other embodiments, the methods of the invention are applicable for adults.

The microbial consortium may be administrated to a human subject by any method known in the art and described herein. In accordance with some embodiments, the methods of the invention comprise administration the microbial consortium or a composition comprising the microbial consortium by oral administration. In accordance with some embodiments, the methods of the invention comprise administration the microbial consortium or a composition comprising the microbial consortium that is formulated for delivery to the intestine. The microbial consortium or a composition comprising the microbial consortium may be formulated for example into a capsule, tablet, food or beverage or any of the like.

In some embodiments, the methods of the invention may comprise administrating the microbial consortium of the invention, compositions or kits comprising the same and optionally an additional treatment, as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month.

The invention further provides the use of a microbial consortium of the invention in the preparation of a composition for treating an inflammatory condition of the gut in a subject in need thereof.

In another aspect, the invention provides a kit comprising microbial consortium of the invention in the preparation of a composition for treating preventing, ameliorating, reducing or delaying the onset of an inflammatory condition in a subject in need thereof. Thus, there is provided a kit comprising two or more microorganisms, at least one of the two or more microorganisms is capable of modulating the production of PE, AEA or a combination thereof. In some embodiments, the kit is for use in treating, preventing, ameliorating, reducing or delaying the onset of an inflammatory condition of the gastrointestinal track.

In some embodiments, the kit is for use in treating, preventing, ameliorating, reducing or delaying the onset of IBD, ulcerative colitis, Crohn's disease or combination thereof.

In some embodiments, the kits described herein may include a composition/suspension as described, as an already prepared dosage form ready for administration or, alternatively, can include the microbial consortium or a composition comprising the microbial consortium as described as a solid pharmaceutical composition (e.g. in a lyophilized form) that can be reconstituted with a solvent to provide a liquid dosage form. The kit of the invention may additionally comprise instructions for using the kit in treating an inflammatory condition of the gut.

The methods of treatment provided herein involve administration of the microbial consortium of the invention in a therapeutically effective amount. The term “effective amount” as used herein is that determined by such considerations as are known to the man of skill in the art.

The present invention provides methods for treating or preventing an inflammatory condition. The term “treatment or prevention” refers to the complete range of therapeutically positive effects of administrating to a subject including inhibition, reduction of, alleviation of, and relief from, an inflammatory condition and specifically an inflammatory condition of the gut or undesired side effects of such an inflammatory condition of the gut related disorders.

As used herein, “disease”, “disorder”, “condition” and the like, as they relate to a subject's health, are used interchangeably and have meanings ascribed to each and all of such terms.

The present invention relates to methods for the treatment of subjects, or patients, in need thereof. By “patient” or “subject in need” it is meant any organism who may be affected by the above-mentioned conditions, and to whom the treatment methods herein described are desired. It should be further noted that particularly in case of human subject, administering of the compositions of the invention to the patient includes both self-administration and administration to the patient by another person. As noted herein above, a patient, an individual or a subject is not limited to a specific age and hence the present invention encompasses children, adults and elderly.

The invention provides methods for treating inflammatory conditions, and further relates to disorders associated or related to inflammatory conditions of the gut. It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology.

According to some embodiments, wherein indicated “increasing” or “enhancing” the activity by means of modulation, as used herein in connection with the microbiome consortium of the invention, it is meant that such increase or enhancement may be an increase or elevation of between about 5% to 100%, specifically, 10% to 100% of the activity as compared to a suitable control for example being no treatment, for example with no stimulant.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

TABLE 1
List of Sequences

| SEQ ID | |
|---|---|
| NO: | Details |
| 1 | Gene encoding 16S rRNA of Clostridium hiranonis DSM |
| | 13275 |
| 2 | Gene encoding 16S rRNA of Anaerostipes hadrus DSM 3319 |
| 3 | Gene encoding 16S rRNA of Bacteroides stercoris CC31F |
| 4 | Gene encoding 16S rRNA of Bacteroides stercoris ATCC |
| | 43183 |
| 5 | Gene encoding 16S rRNA of Phascolarctobacterium |
| | succinatutens YIT 12067 |
| 6 | Gene encoding 16S rRNA of Clostridium bolteae ATCC |
| | BAA-613 |
| 7 | Gene encoding 16S rRNA of Megamonas hypermegale |
| | NCTC10570 |
| 8 | Gene encoding 16S rRNA of Lactococcus lactis subsp. |
| | cremoris MG1363 |
| 9 | Gene encoding 16S rRNA of Clostridium scindens ATCC |
| | 35704 |
| 10 | Gene encoding 16S rRNA of Parabacteroides distasonis |
| | CL09T03C24 |
| 11 | Gene encoding 16S rRNA of Eubacterium limosum strain |
| | SA11 |
| 12 | Gene encoding 16S rRNA of Clostridium sp. SS2/1 |
| 13 | Gene encoding 16S rRNA of Lachnospiraceae bacterium |
| | 5_1_63FAA |

### NON-LIMITING EXAMPLES

### Example 1: In Vitro Studies

### Example 1A: Anandamide (AEA) Synthesis

Materials and methods:

To test this, murine macrophages cell line (RAW cells, 5×10\hat{ }7 cells) were incubated over night with the bacteria Anderostipes hadrus 10\hat{ }8 CFU/ml. Cells were harvested using trypsin, washed in PBS and analyzed for AEA and it one of its precursors, Arachidonic acid (AA) content in HPLC-MS/MS in a positive ion mode using Electron Spray Ionization (ESI).

Cells were extracted with CHCl₃:MeOH (2:1, vol/vol) with [²H4] AEA as internal standard and washed three times with ice-cold CHCl₃, dried under nitrogen flow, and reconstituted with MeOH. Then, total proteins were precipitated. LC-MS/MS analyses were conducted on an AB Sciex (Framingham, MA, USA) Triple Quad 5500 mass spectrometer coupled with a Shimadzu (Kyoto, Japan) UHPLC System. Liquid chromatographic separation was carried out using a Kinetex (Phenomenex) column (C18, 2.6 μm particle size, 100×2.1 mm). Anandamide (AEA) and Arachidonic acid (AA) were detected in a positive ion mode under ESI and MRM conditions. The molecular ions and fragments for each compound were m/z 348.3→62.1 (quantifier) and 91.1 (qualifier) for AEA, m/z 305.2→91.1 (quantifier) and 77.1 (qualifier) for AA, 91.1 (qualifier) for [²H4] AEA. The levels of AEA and AA in the samples were measured against standard curves.

### Results

As can be seen in FIGS. 1A and 1B, treatment with the bacteria Anaerostipes hadrus modulated the levels of both AEA and AA.

FIGS. 1A and 1B, show that in macrophages cells treated with Anderostipes hadrus as compared to AEA level in a control sample (i.e. in the absence of Anaerostipes hadrus), AA levels decreased (FIG. 1A), whereas AEA levels significant increased (FIG. 1B).

AA is one of the precursors for the synthesis of AEA, together with PE. Hence, a reduction in the level of AA correlates with the increase in the level of AEA and suggest use of PE secreted from the Anaerostipes hadrus that participate in AEA synthesis.

These results show that Anderostipes hadrus affected the production of AEA in the host cells (macrophages). It may be suggested that treatment with Anderostipes hadrus has a direct effect on various pathways in the host cells which eventually result in increased AEA production.

### Example 1B: AEA Synthesis

Murine macrophages cell line (RAW cells, 5×10\hat{ }7 cells) are incubated over night with the bacteria Anderostipes hadrus 10\hat{ }8 CFU/ml or commercially available bacterial Phosphatidyl ethanolamine (PE) or a control bacteria (that does not synthesize PE). Cells are harvested using trypsin, washed in PBS and analyzed for Anandamide content in HPLC-MS/MS in a positive ion mode using Electron Spray Ionization (ESI).

### Example 1C: IL-10 Production

Human peripheral blood mononuclear cells (PBMCs) are incubated with each of the microorganisms described herein, for example, in Table 1 above, or a consortium as described herein in 1:1 ratio or with bacterial conditioned media for 72-144 hours. Following this incubation cells media is analyzed for levels of the anti-inflammatory cytokine IL-10 by ELISA and cell population is analyzed by flow cytometry to assess differentiation of T-cells to regulatory T cells (Tregs) using FoxP3+ marker.

### Example 2: in Vivo Studies—Dextran Sodium Sulfate (DSS)—Induced Colitis in Mice Model

The objective of the study was to determine the efficacy of bacterial consortia administration in prevention and treatment of Dextran Sodium Sulfate (DSS)—induced colitis in mice model.

### Materials and Methods:

In this experiment the following bacterial consortia were tested:

The first consortium comprises Clostridium hiranonis DSM 13275, Anaerostipes hadrus DSM 3319, Bacteroides stercoris ATCC 43183 and Megamonas hypermegaleNCTC10570 (denoted herein as “Consortium 1” or “BMC321”)

The second consortium comprises Clostridium hiranonis DSM 13275, Anaerostipes hadrus DSM3319, Clostridium bolteae ATCC BAA-613 and Lactococcus lactis subsp. cremoris MG1363 (denoted herein as “Consortium 2” or “BMC322”)

Mice received one of the above indicated bacterial consortia (BMC321 or BMC322) via oral gavage every other day for 1 week prior to DSS administration followed by another week in parallel to administration of DSS which mice received through their drinking water.

Each bacteria in a consortium was in a concentration of 10^{\hat{ }9} CFU (total 4×10\hat{ }9 CFU bacteria per consortium).

PBS was administrated via oral gavage at similar volume used to administer bacterial consortia to two control group mice (either with or without DSS administration). During the week of DSS administration, stool was collected and analyzed for lipocalin levels by mouse Lipocalin-2/NGAL DouSet ELISA kit (R&D systems) to assess levels of intestinal inflammation. Disease severity was categorized by levels of lipocalin level.

### Results:

Lipocalin-2 is a biomarker for inflammatory disease of the gut and hence the level of lipocalin, for example, in stool can be a measure of an inflammatory process/disease and it's severity.

Higher lipocalin level indicates more severe inflammation.

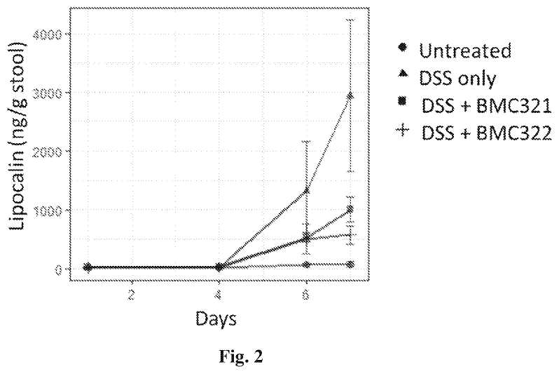

As shown in FIG. 2, in mice administered with DSS alone, an increase in lipocalin levels was observed in mice stool starting from day 4 of the experiment, i.e. after 4 days of DSS administration to the tested mice.

Lower levels of lipocalin were observed in each of the two bacterial consortia-treated groups compared to the untreated group, under the exposure to DSS, from Day 4 of DSS administration up to Day7 (when DSS was stopped). This suggests activation of anti-inflammatory mechanisms by the administered bacterial consortia.

As evident from these results, administration of each one of these two bacteria consortium successfully alleviated/reduced the DSS-induced inflammatory effect.

These results suggested that the bacterial consortia have a preventive role and a therapeutic role.

As noted above, disease severity is associated with increased lipocalin level and hence disease severity can be categorized by lipocalin values. A lipocalin level higher than 1000 ng/g stool was set as a cutoff and the disease severity was calculated following 7 days of DSS administration.

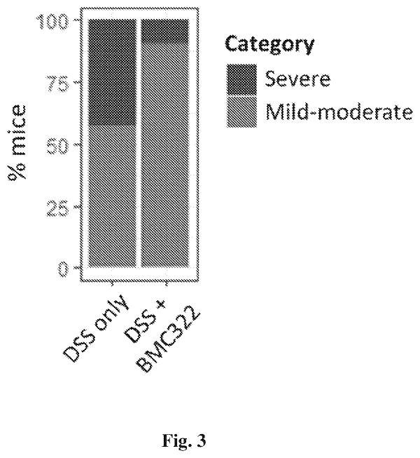

As shown in FIG. 3, after 7 days of administration of DSS alone, 43% of the tested mice in the control group were categorized as having a severe disease.

In contrast, in the group of mice that were treated with BMC322 only 10% mice were categorized as having a sever disease. Thus, these results indicated that treatment with the bacterial consortium BMC322 reduced the disease severity and was effective in allowing the mice to recover from the inflammation.

### Example 3: DSS—Induced Colitis In Mice Model—In Vivo Studies

The objective of the study was to determine the efficacy of bacterial consortia administration on the recovery of DSS-induced colitis in mice model.

### Materials and Methods:

Mice received bacterial consortia (BMC322 as detailed above) via oral gavage every other day for 1 week in parallel to administration of DSS, which mice received through their drinking water. Each bacteria in a consortium was in a concentration of 10\hat{ }9 CFU (total 4×10\hat{ }9 CFU bacteria per consortium).

Following DSS cessation, mice continued to receive oral gavage of bacterial consortia for another week. PBS was administrated via oral gavage at similar volume used to administer bacterial consortia to two control group mice (either with or without DSS administration).

Monitoring of the mice continued for 20 days after the beginning of DSS administration. Disease severity was assessed by Disease Activity Index (DAI) scale which evaluates stool consistency, rectal bleeding and change in body weight, each in a separate score and combined them to a weighted score.

During the week of DSS administration and the week after, stool was collected and analyzed for Lipocalin levels by mouse Lipocalin-2/NGAL DouSet ELISA kit (R&D systems) to assess levels of intestinal inflammation.

Mice that lost more than 20% of their initial body weight were removed from study due to humane reasons. Survival of mice in the different groups was evaluated using the Kaplan-Meyer survival analysis.

Upon study termination, spleen weight and colon length were measured as a parameter of inflammation severity.

### Results

As noted above in connection with Example 2, lipocalin-2 levels, for example, in stool can be a measure of an inflammatory process/disease and its severity.

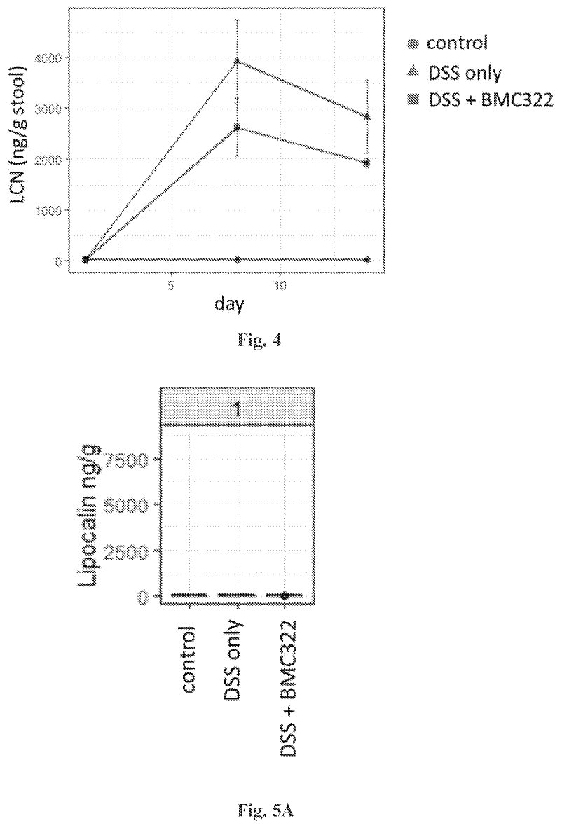

As shown in FIG. 4, in mice administered with DSS alone, an increase in lipocalin levels in mice stool was observed starting immediately after DSS administration to the tested mice with a maximal value of lipocalin at day 8 of the experiment (2 days after cessation of DSS administration).

A reduction in lipocalin levels was observed in stool of mice that were treated with the bacterial consortia. These results indicate that the therapeutic effect of the bacteria on the inflammatory disease.

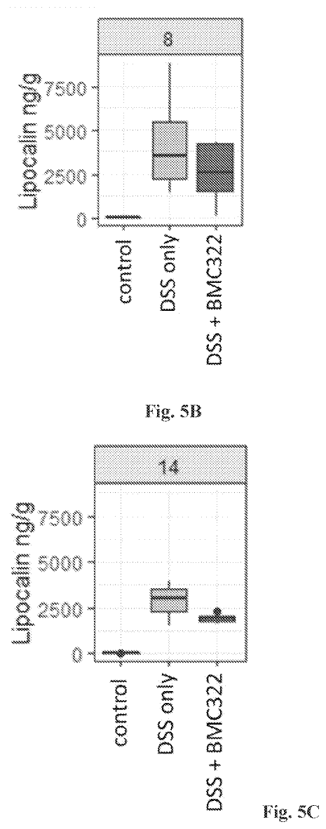

FIGS. 5A to 5C show the median lipocalin levels in stool of the mice administrated with DSS with or without the bacteria. As shown, there was a reduction in lipocalin levels in the mice treated with the bacterial consortium, denoted herein as BMC322 for 14 days (FIG. 5C).

Hence, these results suggest a therapeutic effect of the tested bacterial consortium on the DSS-induced colitis.

The effect of DSS induced colitis was also evaluated by measuring several parameters including stool consistency, rectal bleeding and mice body weight.

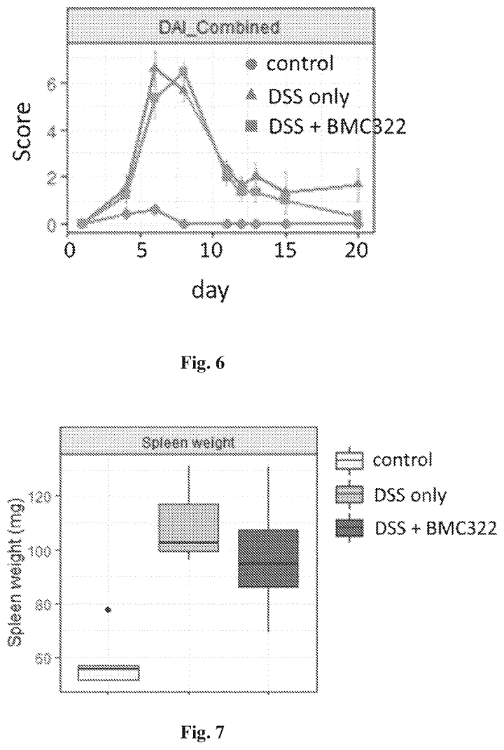

The results in FIG. 6 show that an improvement was observed staring from the 15 day of the experiment in mice treated with the bacterial consortium, denoted herein as BMC322.

The severity of the inflammatory process was evaluated by additional measures such as spleen weight and colon length. An increase in spleen weight and a reduction in colon length are considered as measures of a severe inflammation.

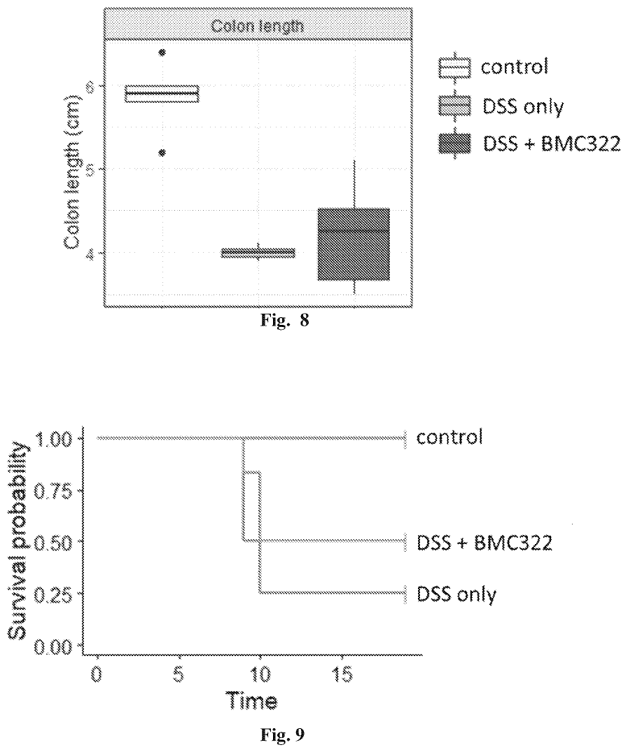

FIGS. 7 and 8 show the effect of bacteria treatment on the spleen weight and colon length, respectively at day 20, at termination. As can be seen in FIG. 7, a reduction in the median spleen weight was observed in the treated mice as compared with the non-treated mice. In addition, and as can be seen in FIG. 8, an increase in the median colon length was observed in the treated mice as compared with the non-treated mice. Enlarged spleen and shortening of the colon indicate higher disease severity.

Taken collectively, the results indicate that treatment with bacterial consortium reduced the severity of the DSS-induced colitis.

Further, the survival probability of treated and not treated mice was evaluated by Kaplen-Meier analysis. The Kaplan-Meier estimator is a non-parametric statistic used to estimate the survival function from lifetime data.

As can be seen in FIG. 9 an increased survival probability was observed in the treated mice, suggesting a protective effect of treatment with bacterial consortium.

### Example 4: Mouse Model of IBD—In Vivo Studies

The objective of the study is to assess the effect of oral administration of two microbial consortia on the reduction of intestinal inflammation in an immunological experimental mouse model of IBD.

In this study, fresh microbial consortia are administered to mice in a concentration of 10\hat{ }9 CFU per strain (total 4×10\hat{ }9 CFU per consortia). The model that is being utilized in this study is genetically modified animal model of Interleukin-10 (IL-10) gene deficient mice. IL-10 is a known regulatory cytokine with IBD susceptibility genes in humans and thus, IL-10 deficient mice model is commonly used for assessment of new therapeutic approaches in IBD. IL-10 deficient mice with a germ-free (GF) background, do not develop spontaneous colitis, which should be induced for an adequate IBD model. Induction of colitis is done by fecal material transplant (FMT) of the animals with feces obtained from human IBD patients. IL-10 deficient GF mice are administered with microbial consortia by oral gavage starting 1 week prior to FMT and proceed at least 4 weeks after FMT. Clinical assessment is carried out by DAI scoring system and fecal lipocalin levels. Mice are monitored for up to 6 weeks after FMT. Luminal content (both fecal and ceacal) will is collected for microbial analysis and lipocalin measurement at termination. Histology of ceacum, distal colon, proximal colon and rectum is performed.

Cytokine levels using cultured colon tissue (e.g. IL-12p40, IFNγ) is assessed by ELISA and gene expression in colon/ceacum tissue (assess levels of cytokines, chemokines, epithelial markers, etc.) is measured and quantified by qPCR.

1-50. (canceled)

51. A method of treating an inflammatory condition in a human subject in need thereof, the method comprising administering to the human subject a therapeutically effective amount of a microbial consortium comprising Clostridium hiranonis and Anaerostipes hadrus, wherein said Anaerostipes hadrus increases production of anandamide (AEA) in the human subject.

52. The method of claim 51, wherein said microbial consortium further comprises at least one microorganism selected from the group consisting of Bacteroides stercoris, Megamonas hypermegale, Clostridium bolteae and Lactococcus lactis.

53. The method of claim 51, wherein said microbial consortium comprises Clostridium hiranonis, Anaerostipes hadrus, Bacteroides stercoris and Megamonas hypermegale.

54. The method of claim 51, wherein said microbial consortium comprises Clostridium hiranonis, Anaerostipes hadrus, Clostridium bolteae and Lactococcus lactis.

55. The method of claim 51, wherein said microbial consortium comprises Clostridium hiranonis, Anaerostipes hadrus, Bacteroides stercoris and Clostridium bolteae.

56. The method of claim 51, wherein said Clostridium hiranonis comprises a 16S rRNA sequence with at least 95% sequence identity to SEQ ID NO: 1 and wherein said Anaerostipes hadrus comprises a 16S rRNA sequence with at least 95% sequence identity to SEQ ID NO: 2.

57. The method of claim 52, wherein said Bacteroides stercoris comprises a 16S rRNA sequence with at least 95% sequence identity to SEQ ID NO: 4, wherein said Megamonas hypermegale comprises a 16S rRNA sequence with at least 95% sequence identity to SEQ ID NO: 7, wherein said Clostridium bolteae comprises a 16S rRNA sequence with at least 95% sequence identity to SEQ ID NO: 6, and wherein said Lactococcus lactis comprises a 16S rRNA sequence with at least 95% sequence identity to SEQ ID NO: 8.

58. The method of claim 51, wherein said microbial consortium comprises Clostridium hiranonis DSM 13275 and Anaerostipes hadrus DSM 3319.

59. The method of claim 58, wherein said microbial consortium further comprises at least one microorganism selected from the group consisting of Bacteroides stercoris ATCC 43183, Megamonas hypermegale NCTC10570, Clostridium bolteae ATCC BAA-613, and Lactococcus lactis subsp. cremoris MG1363.

60. The method of claim 51, wherein said microbial consortium comprises Clostridium hiranonis DSM 13275, Anaerostipes hadrus DSM 3319, Bacteroides stercoris ATCC 43183 and Megamonas hypermegale NCTC10570.

61. The method of claim 51, wherein said microbial consortium comprises Clostridium hiranonis DSM 13275, Anaerostipes hadrus DSM 3319, Clostridium bolteae ATCC BAA-613 and Lactococcus lactis subsp. cremoris MG1363.

62. The method of claim 51, wherein said microbial consortium comprises Clostridium hiranonis DSM 13275, Anaerostipes hadrus DSM 3319, Bacteroides stercoris ATCC 43183 and Clostridium bolteae ATCC BAA-613.

63. The method of claim 51, wherein said inflammation condition is an inflammatory condition of the gastrointestinal tract.

64. The method of claim 63, wherein the inflammatory condition of the gastrointestinal tract is one or more of Crohn's disease, inflammatory bowel disease, gastritis, colitis, ulcerative colitis, irritable bowel syndrome, gastric ulcer, or duodenal ulcer.

65. The method of claim 64, wherein the inflammatory condition of the gastrointestinal tract is one or more of inflammatory bowel disease (IBD), ulcerative colitis or Crohn's disease.

66. The method of claim 65, wherein the inflammatory condition of the gastrointestinal tract is IBD.
